(19) United States
(12) Patent Application Publication
(10) Pub. No.: US 2023/0117048 A1
(43) Pub. Date: Apr. 20, 2023

(54) STEM CELL COMPOSITIONS AND METHODS OF REPAIRING TISSUE

(71) Applicant: SMSBIOTECH, INC., San Marcos, CA (US)

(72) Inventor: Abdulkader Rahmo, San Marcos, CA (US)

(21) Appl. No.: 17/905,851

(22) PCT Filed: Feb. 24, 2021
(86) PCT No.: PCT/US21/19446

§ 371 Date: Sep. 8, 2022

Related U.S. Application Data
(60) Provisional application No. 62/987,270, filed on Mar. 9, 2020.

(51) Int. Cl. A61K 35/545; A61P 17/02

(52) U.S. Cl. CPC A61K 35/545; A61P 17/02

(57) ABSTRACT

Disclosed herein are compositions for use in promoting or improving repair or regeneration of damaged tissue or for improving recovery from a viral, bacterial or fungal infection. The compositions include small mobile stem (SMS) cells. Also disclosed are methods of making the compositions, and methods of using the compositions for treating or ameliorating inflammatory illnesses, such as fibrosis or COPD, and/or a viral, bacterial or fungal infection or the damage caused by such pathogens.

### CROSS-REFERENCE TO RELATED APPLICATION

This application claims the benefit of priority of U.S. Provisional Application 62/987,270, filed Mar. 9, 2020, which is hereby expressly incorporated by reference in its entirety.

### FIELD

Aspects of the present disclosure relate to compositions and methods for promoting repair or regeneration of damaged tissue or organs, and for mitigating parenchymal injury. In some aspects, the compositions include isolated small mobile stems cells, and the compositions and methods are used for treating or ameliorating a viral, bacterial or fungal infection, or the damage caused by a viral, bacterial or fungal infection by repairing tissue resulting from the infection, or treating or ameliorating the adverse effects caused by inflammatory disease, such as fibrosis or COPD, and/or an inflammatory disease associated with a viral, bacterial or fungal infection.

### BACKGROUND

The process of inflammation, while important for clearing of pathogens, has been associated with a wide range of clinical disorders. In some cases, the inflammatory response exhibited by an individual can be more deleterious than the originating stimulus, including proinflammatory formation of fibrotic scar tissue that interferes with normal organ function. In other cases, abnormal autoinflammation and chronic inflammation often leads to life-long and uncurable disorders or conditions associated with tissue degeneration of the individual.

Virus infections remain a major health problem worldwide with annual epidemics, which are often complicated by significant morbidity and mortality despite the availability of rationalized vaccination protocols based on the use of an inactivated virus. Vaccines have several drawbacks, including: the protection induced by the vaccine is short-lived and therefore requires reoccurring administration; the vaccine is only capable of eliciting a strain-specific antibody response; and the vaccine is incapable of restoring or repairing damaged tissues or organs from the viral infection. Viral strains can be highly contagious and continuously variable because of antigenic shift, which occurs as a result of RNA segments exchanged between virus strains and antigenic drift due to point mutations, thereby exacerbating these drawbacks.

Coronaviruses are a family of host-specific enveloped RNA viruses with a single-stranded positive sense genome. The novel coronavirus first isolated in Wuhan, China, had more infections than the previous severe acute respiratory syndrome (SARS) outbreak of 2002 and 2003, and results in coronavirus disease 2019 (COVID-19). The most severe form of the infection is Severe Acute Respiratory Syndrome Coronavirus 2 (SARS-CoV-2), characterized as acute respiratory compromise followed by acute respiratory failure, with relatively high morbidity and mortality secondary to irreversible pulmonary tissue injury and fibrotic scarring, despite intense supportive medical care. The pathophysiology of the severe respiratory syndrome is multifactorial, manifesting with hyper-immune response and concomitant inflammatory cytokine storm (associated with other end organ impairment), lysis of alveolar and bronchial epithelial cells, and often secondary opportunistic pneumonia. Specific antiviral therapies and vaccines for SARS-CoV-2 treatment and prophylaxes are only beginning to be developed and approved. Furthermore, there is an ongoing concern of SARS-CoV-2 variants, which may be more virulent and less susceptible to current therapies. As such, critical care is still essential and consists of treating symptoms, utilizing mechanical ventilation with endo-tracheal intubation, extracorporeal membrane oxygenation (ECMO), vasopressor medications to treat shock, as well as antibiotics for opportunistic pneumonias that may develop. It has become clear that patients with advancing age and co-morbidities such as diabetes and heart disease are likely to experience poor outcomes if they develop SARS-CoV-2, including long-term debility if they do recover, permanent pulmonary impairment, or death. This population has an estimated mortality rate of approximately 10%. Therefore, it is vital to develop safe and effective therapeutic approaches for patients with severe coronavirus disease.

### SUMMARY

The present disclosure relates generally to compositions for promoting or improving repair or regeneration of damaged tissue or for improving recovery from a viral, fungal, or bacterial infection, or a sequela thereof, and methods of making and using the same. The compositions include small mobile stem (SMS) cells, which may be allogeneic or autologous, and which may be derived from peripheral blood. The viral, fungal, or bacterial infection may include SARS-CoV-2 infection.

Accordingly, some embodiments provided herein relate to methods of promoting or improving repair or regeneration of damaged tissue or for improving recovery from a viral, fungal, or bacterial infection or a sequela thereof, comprising selecting a subject suffering from a viral, fungal, or bacterial infection or the sequela thereof, and administering to the subject a composition comprising a therapeutically effective amount of small mobile stem (SMS) cells. In some embodiments, a subject is selected who does not have a viral, fungal, or bacterial infection or a sequela thereof, but is administered the compositions provided herein in order to prevent tissue damage in the instance that a viral, fungal, or bacterial infection were to take place. In some embodiments, the SMS cells are obtained from peripheral blood. In some embodiments, the SMS cells are allogenic. In some embodiments, the SMS cells are autologous. In some embodiments, the damaged tissue is lung tissue. In some embodiments, wherein administering the compositions reduces or inhibits alveolar cell injury, reduces or inhibits respiratory endothelial cell injury, increases or improves the repair of damaged lung tissue, increases or enhances the regeneration of damaged lung tissue, or reduces or inhibits development of pulmonary fibrosis. In some embodiments, the damaged tissue is a result of the viral, fungal, or bacterial infection or a sequela thereof. In some embodiments, the viral infection is caused by a single-stranded RNA virus, a double-stranded RNA virus, a positive-sense ssRNA virus, a negative-sense ssRNA virus, a double-stranded DNA virus, or a single-stranded DNA virus. In some embodiments, the viral, fungal, or bacterial infection or the sequela thereof is caused by a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, Filovirus, coxsackievirus, equine encephalitis virus, a Rift Valley fever virus, an alphavirus, hepatitis A virus, hepatitis B virus, hepatitis C virus, hepatitis D virus, or a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof. In some embodiments, the coronavirus is a MERS-CoV, SARS-CoV, or SARS-CoV-2. In some embodiments, the coronavirus is SARS-CoV-2. In some embodiments, the composition comprises aerosolized SMS cells, which is, optionally, administered via inhalation e.g., via the nose or mouth. In some embodiments, the composition is administered intravenously. In some embodiments, the SMS cells are administered in combination with an antibiotic, such as azithromycin, an antifungal, such as Voriconazole, Amphotericin B or itraconazole, a vaccine or an anti-viral medication, such as remdesivir. In some embodiments, the vaccine is a vaccine against one or more of coronavirus, cholera, dengue, diphtheria, Haemophilus influenza type b infection, hepatitis A, hepatitis B, influenza, Japanese encephalitis, meningococcal meningitis, pertussis, polio, rabies, tetanus, tuberculosis, typhoid, or yellow fever. In some embodiments, the vaccine is a coronavirus COVID-19 vaccine.

Some embodiments described herein relate to methods of treating or inhibiting an inflammatory disease. In some embodiments, the methods comprise selecting a subject having the inflammatory disease and administering to the selected subject a composition comprising a therapeutically effective amount of small mobile stem (SMS) cells. In some embodiments, the inflammatory disease is a pulmonary inflammatory disease. In some embodiments, the inflammatory disease comprises fibrosis. In some embodiments, the fibrosis is pulmonary fibrosis, liver fibrosis, kidney fibrosis, or cardiac fibrosis. In some embodiments, the inflammatory disease comprises chronic pulmonary obstructive disease (COPD). In some embodiments, the inflammatory disease comprises acute respiratory distress syndrome (ARDS). In some embodiments, the inflammatory disease is associated with or caused by a viral, fungal and/or bacterial infection. In some embodiments, the viral infection is caused by a single-stranded RNA virus, a double-stranded RNA virus, a positive-sense ssRNA virus, a negative-sense ssRNA virus, a double-stranded DNA virus, or a single-stranded DNA virus. In some embodiments, the viral, fungal, or bacterial infection is caused by a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, filovirus, coxsackievirus, equine encephalitis virus, a Rift Valley fever virus, an alphavirus, hepatitis A virus, hepatitis B virus, hepatitis C virus, hepatitis D virus, or a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof. In some embodiments, the viral infection is a coronavirus infection. In some embodiments, the viral infection is a SARS-CoV, MERS-CoV, or SARS-CoV-2 infection. In some embodiments, the SMS cells are obtained from peripheral blood. In some embodiments, the SMS cells are allogeneic or autologous to the subject. In some embodiments, administering the composition reduces or mitigates alveolar cell injury, reduces or mitigates respiratory endothelial cell injury, increases or improves the repair of damaged lung tissue, increases or enhances regeneration of damaged lung tissue, or is associated with a reduced or inhibited development of pulmonary fibrosis, or any combination thereof. In some embodiments, the composition is formulated for administration by inhalation. In some embodiments, the composition is formulated for intravenous administration. In some embodiments, the composition is administered with a standard of care treatment for the inflammatory disease. In some embodiments, the inflammatory disease is associated with a viral, fungal, or bacterial infection and the standard of care treatment is an antibiotic, such as azithromycin, an antifungal, such as Voriconazole, Amphotericin B or itraconazole, a vaccine or an anti-viral medication, such as dexamethasone, convalescent serum, tocilizumab, sarilumab, ribavirin, favapiravir, darunavir, galidesivir, interferon alpha, interferon beta, lopinavir, ritonavir, remdesivir, triazavirin, umifenovir, or any combination thereof. In some embodiments, the vaccine is a vaccine against one or more of coronavirus, cholera, dengue, diphtheria, Haemophilus influenza type b infection, hepatitis A, hepatitis B, influenza, Japanese encephalitis, meningococcal meningitis, pertussis, polio, rabies, tetanus, tuberculosis, typhoid, or yellow fever. In some embodiments, the vaccine is a coronavirus COVID-19 vaccine.

Some embodiments provided herein relate to compositions comprising a therapeutically effective amount of small mobile stem (SMS) cells. In some embodiments, the compositions are formulated for use in repairing or regenerating damaged tissue or for improving recovery from a viral, fungal, or bacterial infection (or a sequela thereof), such as a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, filovirus, coxsackievirus, equine encephalitis virus, a Rift Valley fever virus, an alphavirus, hepatitis A virus, hepatitis B virus, hepatitis C virus, hepatitis D virus, or a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof, preferably MERS-CoV, SARS-CoV, or SARS-CoV-2. In some embodiments, the SMS cells are obtained from peripheral blood. In some embodiments, the composition comprises aerosolized SMS cells, which are, optionally, formulated for inhalable administration. In some embodiments, the composition is formulated for intravenous administration. In some embodiments, the damaged tissue is caused by a viral infection. In some embodiments, the viral infection is caused by a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, filovirus, coxsackievirus, equine encephalitis virus, a Rift Valley fever virus, an alphavirus, hepatitis A virus, hepatitis B virus, hepatitis C virus, hepatitis D virus, or hepatitis E virus, or a combination thereof. In some embodiments, the coronavirus is SARS-CoV-2. In some embodiments, the therapeutically effective amount is an amount sufficient to cause reduced or inhibited alveolar cell injury, reduced or inhibited respiratory endothelial cell injury, reduced or inhibited development of pulmonary fibrosis, increased or improved repair of damaged lung tissue, or increased or enhanced regeneration of damaged lung tissue. In some embodiments, the compositions further include a therapeutically effective amount of an antibiotic, antifungal, vaccine or an anti-viral medication, such as remdesivir. In some embodiments, the vaccine is a coronavirus vaccine. In some embodiments, the compositions further include a pharmaceutically acceptable carrier, preservative, antioxidant, diluent, or excipient, or any combination thereof

Some embodiments provided herein relate to uses of any of the compositions described herein for treating or inhibiting a viral, fungal, or bacterial infection (or a sequela thereof), such as a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, Filovirus, coxsackievirus, equine encephalitis virus, a Rift Valley fever virus, an alphavirus, hepatitis A virus, hepatitis B virus, hepatitis C virus, hepatitis D virus, or a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof, preferably MERS-CoV, SARS-CoV, or SARS-CoV-2. Some embodiments provided herein relate to any of the compositions described herein for use in a medicament.

Some embodiments provided herein relate to uses of any of the compositions disclosed herein for the treatment, inhibition, amelioration, or mitigation of an inflammatory disease. In some embodiments, the inflammatory disease is pulmonary inflammatory disease. In some embodiments, the inflammatory disease comprises fibrosis. In some embodiments, the fibrosis is pulmonary fibrosis, liver fibrosis, kidney fibrosis, or cardiac fibrosis. In some embodiments, the inflammatory disease comprises chronic pulmonary obstructive disease (COPD). In some embodiments, the inflammatory disease comprises acute respiratory distress syndrome (ARDS). In some embodiments, the inflammatory disease is associated with a viral, fungal, or bacterial infection. In some embodiments, the viral infection is a coronavirus infection. In some embodiments, the viral infection is a SARS-CoV, MERS-CoV, or SARS-CoV-2 infection.

Accordingly, some aspects of the embodiments described herein relate to the following numbered alternatives:

1. A method of promoting or improving repair or regeneration of damaged tissue or for improving recovery from a viral, fungal, or bacterial infection or a sequela thereof, comprising: selecting a subject that has or has had a viral, fungal, or bacterial infection or the sequela thereof; and administering to the selected subject a composition comprising a therapeutically effective amount of small mobile stem (SMS) cells.

2. The method of alternative 1, wherein the SMS cells are obtained from peripheral blood.

3. The method of any one of alternatives 1-2, wherein the SMS cells are allogenic.

4. The method of any one of alternatives 1-2, wherein the SMS cells are autologous.

5. The method of any one of alternatives 1-4, wherein the damaged tissue is lung tissue.

6. The method of alternative 5, wherein administering the compositions reduces or inhibits alveolar cell injury, reduces or inhibits respiratory endothelial cell injury, increases or improves the repair of damaged lung tissue, increases or enhances the regeneration of damaged lung tissue, or reduces or inhibits the development of pulmonary fibrosis.

7. The method of any one of alternatives 1-6, wherein the damaged tissue is a result of the viral, bacterial, or fungal infection.

8. The method of any one of alternatives 1-7, wherein the viral infection is caused by a single-stranded RNA virus, a double-stranded RNA virus, a positive-sense ssRNA virus, a negative-sense ssRNA virus, a double-stranded DNA virus, or a single-stranded DNA virus.

9. The method of any one of alternatives 1-7, wherein the viral, fungal, or bacterial infection or the sequela thereof is caused by a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, Filovirus, coxsackievirus, equine encephalitis virus, a Rift Valley fever virus, an alphavirus, hepatitis A virus, hepatitis B virus, hepatitis C virus, hepatitis D virus, or a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof

10. The method of alternative 9, wherein the coronavirus is a MERS-CoV, SARS-CoV, or SARS-CoV-2.

11. The method of alternative 9, wherein the coronavirus is SARS-CoV-2.

12. The method of any one of alternatives 1-11, wherein the composition comprises aerosolized SMS cells, which is, optionally, administered via inhalation.

13. The method of any one of alternatives 1-11, wherein the composition is administered intravenously.

14. The method of any one of alternatives 1-13, wherein the SMS cells are administered in combination with an antibiotic, such as azithromycin, an antifungal, such as Voriconazole, Amphotericin B or itraconazole, a vaccine or an anti-viral medication, such as remdesivir.

15. The method of alternative 14, wherein the vaccine is a vaccine against one or more of coronavirus, cholera, dengue, diphtheria, Haemophilus influzenzoe type b infection, hepatitis A, hepatitis B, influenza, Japanese encephalitis, meningococcal meningitis, pertussis, polio, rabies, tetanus, tuberculosis, typhoid, or yellow fever.

16. The method of any one of alternatives 14-15, wherein the vaccine is a coronavirus COVID-19 vaccine.

17. The method of any one of alternatives 1-16, wherein administration of the composition to the selected subject upregulates Dickkopf-related protein 1 (DKK1), N-acylglucosamine 2-epimerase (RENBP), growth/differentiation factor 15 (GDF15), dermokine (DMKN), ferritin, light (FTL), tryptophan 2,3-dioxygenase (TDO2), apolipoprotein E (APOE), chitinase-3-like protein 1 (CHI3L1), or nuclear receptor subfamily 4 group A member 2 (NR4A2), or any combination thereof, in the selected subject.

18. The method of any one of alternatives 1-17, wherein administration of the composition to the selected subject downregulates interleukin-11 (IL-11), SPRY domain-containing SOCS box protein 1 (SPSB1), cytochrome P450 26B1 (CYP26B1), or frizzled-8 (FZD8), or any combination thereof, in the selected subject.

19. A composition comprising a therapeutically effective amount of small mobile stem (SMS) cells for use in repairing or regenerating damaged tissue, such as lung tissue, or for improving recovery from a viral, fungal, or bacterial infection or a sequela thereof, such as a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, Filovirus, coxsackievirus, equine encephalitis virus, a Rift Valley fever virus, an alphavirus, hepatitis A virus, hepatitis B virus, hepatitis C virus, hepatitis D virus, or a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof, preferably MERS-CoV, SARS-CoV, or SARS-CoV-2, in a subject in need thereof.

20. The composition of alternative 19, wherein the SMS cells are obtained from peripheral blood.

21. The composition of any one of alternatives 19 or 20, wherein the composition comprises aerosolized SMS cells, which are, optionally, formulated for inhalable administration.

22. The compositions of any one of alternatives 19-21, wherein the composition is formulated for intravenous administration.

23. The composition of any one of alternatives 19-22, wherein the damaged tissue is caused by a viral infection.

24. The composition of alternative 23, wherein the viral infection is caused by a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, Filovirus, coxsackievirus, equine encephalitis virus, a Rift Valley fever virus, an alphavirus, hepatitis A virus, hepatitis B virus, hepatitis C virus, hepatitis D virus, or hepatitis E virus, or a combination thereof

25. The composition of alternative 24, wherein the coronavirus is SARS-CoV-2.

26. The composition of any one of alternatives 19-25, wherein the therapeutically effective amount is an amount sufficient to cause reduced alveolar cell injury, reduced respiratory endothelial cell injury, reduced development of pulmonary fibrosis, increased repair of damaged lung tissue, or increased regeneration of damaged lung tissue.

27. The composition of any one of alternatives 19-26, further comprising a therapeutically effective amount of an antibiotic, such as azithromycin, an antifungal, such as Voriconazole, Amphotericin B or itraconazole, a vaccine or an anti-viral medication, such as remdesivir.

28. The composition of alternative 27, wherein the vaccine is a coronavirus vaccine.

29. The composition of any one of alternatives 19-28, further comprising a pharmaceutically acceptable carrier, preservative, antioxidant, diluent, or excipient, or any combination thereof

30. The composition of any one of alternatives 19-29, wherein the composition upregulates Dickkopf-related protein 1 (DKK1), N-acylglucosamine 2-epimerase (RENBP), growth/differentiation factor 15 (GDF15), dermokine (DMKN), ferritin, light (FTL), tryptophan 2,3-dioxygenase (TDO2), apolipoprotein E (APOE), chitinase-3-like protein 1 (CHI3L1), or nuclear receptor subfamily 4 group A member 2 (NR4A2), or any combination thereof, in the subject in need thereof

31. The composition of any one of alternatives 19-30, wherein the composition downregulates interleukin-11 (IL-11), SPRY domain-containing SOCS box protein 1 (SPSB1), cytochrome P450 26B1 (CYP26B1), or frizzled-8 (FZD8), or any combination thereof, in the subject in need thereof

32. Use of the composition of any one of alternatives 19-31 for treating or inhibiting a viral, fungal, or bacterial infection, such as a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, filovirus, coxsackievirus, equine encephalitis virus, a Rift Valley fever virus, an alphavirus, hepatitis A virus, hepatitis B virus, hepatitis C virus, hepatitis D virus, or a hepatitis E virus, pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof, preferably MERS-CoV, SARS-CoV, or SARS-CoV-2, or the tissue damage resulting from said infection.

33. The composition of any one of alternatives 19-31 for use in a medicament.

34. The composition of any one of alternatives 19-31 for use in the treatment or inhibition of an inflammatory disease.

35. The composition of alternative 34, wherein the inflammatory disease is a pulmonary inflammatory disease.

36. The composition of alternative 34 or 35, wherein the inflammatory disease comprises fibrosis, such as pulmonary fibrosis, liver fibrosis, kidney fibrosis, or cardiac fibrosis.

37. The composition of any one of alternatives 34-36, wherein the inflammatory disease comprises chronic pulmonary obstructive disease (COPD).

38. The composition of any one of alternatives 34-37, wherein the inflammatory disease comprises acute respiratory distress syndrome (ARDS).

39. The composition of any one of alternatives 34-38, wherein the inflammatory disease is associated with or caused by a viral, fungal, or bacterial infection.

40. The composition of alternative 39, wherein the viral infection is a coronavirus infection.

41. The composition of alternative 39 or 40, wherein the viral infection is a SARS-CoV, MERS-CoV, or SARS-CoV-2 infection.

42. A method of treating or inhibiting an inflammatory disease, comprising:

selecting a subject suffering from the inflammatory disease; and

administering to the selected subject a composition comprising a therapeutically effective amount of small mobile stem (SMS) cells.

43. The method of alternative 42, wherein the inflammatory disease is a pulmonary inflammatory disease.

44. The method of alternative 42 or 43, wherein the inflammatory disease comprises fibrosis, such as pulmonary fibrosis, liver fibrosis, kidney fibrosis, or cardiac fibrosis.

45. The method of any one of alternatives 42-44, wherein the inflammatory disease comprises chronic pulmonary obstructive disease (COPD).

46. The method of any one of alternatives 42-45, wherein the inflammatory disease comprises acute respiratory distress syndrome (ARDS).

47. The method of any one of alternatives 42-46, wherein the inflammatory disease is associated with a viral, fungal, or bacterial infection.

48. The method of alternative 47, wherein the viral infection is caused by a single-stranded RNA virus, a double-stranded RNA virus, a positive-sense ssRNA virus, a negative-sense ssRNA virus, a double-stranded DNA virus, or a single-stranded DNA virus.

49. The method of alternative 47 or 48, wherein the viral, fungal, or bacterial infection is caused by a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, Filovirus, coxsackievirus, equine encephalitis virus, a Rift Valley fever virus, an alphavirus, hepatitis A virus, hepatitis B virus, hepatitis C virus, hepatitis D virus, a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof

50. The method of any one of alternatives 47-49, wherein the viral infection is a coronavirus infection.

51. The method of any one of alternatives 47-50, wherein the viral infection is a SARS-CoV, MERS-CoV, or SARS-CoV-2 infection.

52. The method of any one of alternatives 42-51, wherein the SMS cells are obtained from peripheral blood.

53. The method of any one of alternatives 42-52, wherein the SMS cells are allogeneic or autologous to the subject.

54. The method of any one of alternatives 42-53, wherein administering the composition reduces or inhibits alveolar cell injury, reduces or inhibits respiratory endothelial cell injury, increases or improves the repair of damaged lung tissue, increases or enhances the regeneration of damaged lung tissue, or reduces or inhibits the development of pulmonary fibrosis, or any combination thereof

55. The method of any one of alternatives 42-54, wherein the composition comprises aerosolized SMS cells, which is, optionally, administered via inhalation.

56. The method of any one of alternatives 42-55, wherein the composition is formulated for administration by inhalation e.g., by the nose or mouth.

57. The method of any one of alternatives 42-56, wherein the composition is formulated for intravenous administration.

58. The method of any one of alternatives 42-57, wherein the composition is administered with a standard of care treatment for the inflammatory disease.

59. The method of alternative 58, wherein the inflammatory disease is associated with or caused by a viral infection and the standard of care treatment is an antibiotic such as azithromycin, an antifungal such as Voriconazole, Amphotericin B or itraconazole, a vaccine or an anti-viral medication, such as remdesivir.

60. The method of alternative 59, wherein the vaccine is a vaccine against one or more of coronavirus, cholera, dengue, diphtheria, Haemophilus influenza type b infection, hepatitis A, hepatitis B, influenza, Japanese encephalitis, meningococcal meningitis, pertussis, polio, rabies, tetanus, tuberculosis, typhoid, or yellow fever.

61. The method of alternative 59 or 60, wherein the vaccine is a coronavirus COVID-19 vaccine.

62. The method of any one of alternatives 42-61, wherein administration of the composition upregulates Dickkopf-related protein 1 (DKK1), N-acylglucosamine 2-epimerase (RENBP), growth/differentiation factor 15 (GDF15), dermokine (DMKN), ferritin, light (FTL), tryptophan 2,3-dioxygenase (TDO2), apolipoprotein E (APOE), chitinase-3-like protein 1 (CHI3L1), or nuclear receptor subfamily 4 group A member 2 (NR4A2), or any combination thereof, in the subject.

63. The method of any one of alternatives 42-62, wherein administration of the composition downregulates interleukin-11 (IL-11), SPRY domain-containing SOCS box protein 1 (SPSB1), cytochrome P450 26B1 (CYP26B1), or frizzled-8 (FZD8), or any combination thereof, in the subject.

### BRIEF DESCRIPTION OF DRAWINGS

In addition to the features described above, additional features and variations will be readily apparent from the following descriptions of the drawings and exemplary embodiments. It is to be understood that these drawings depict typical embodiments and are not intended to be limiting in scope.

FIGS. 1A-C depict SMS cells adhering to and improving proliferation of human adipose derived mesenchymal stem cells. FIG. 1A depicts green fluorescence stained SMS cells co-cultured with human adipose derived mesenchymal stem cells (image at 100×). FIG. 1B depicts flow cytometric detection of green fluorescence in human adipose derived mesenchymal stem cells with attached fluorescence-stained SMS cells. Mesenchymal stem cells incubated with SMS cells resulted in 89% live cells counted, while control mesenchymal stem cells resulted in 76% live cells counted. FIG. 1C depicts the total number of cells and live cells for both treated (with SMS cells) and non-treated human adipose derived mesenchymal stem primary cells (Ad-MSC) flasks. SMS cells were added to flasks (labeled “treated”) of Ad-MSC at ˜50% confluency. The non-treated flask was cultured under the same conditions, except without SMS cells. The total number of cells includes both dead and live cells. The bars on the graph represent the average number taken from 4 measurements. The error bars represent standard deviation of measurements within each population.

FIGS. 2A-C depict SMS cells adhering to and inhibiting proliferation of human dermal fibroblasts. FIG. 2A depicts green fluorescence stained SMS cells co-cultured with human fibroblasts (image at 100×). FIG. 2B depicts flow cytometric detection of green fluorescence in human fibroblasts with attached fluorescence-stained SMS cells. Fibroblasts incubated with SMS cells resulted in 90% live cells counted, while control fibroblasts resulted in 89% live cells counted. FIG. 2C depicts total number of viable fibroblasts as a function of SMS cell concentration in co-culture. The viabilities of fibroblasts were determined using Trypan blue exclusion and were calculated using the Countess automated cell counter (Invitrogen). The size gating parameters were set at 5μm and 20 μm.

FIGS. 3A-C depict SMS cells adhering to and improving proliferation of human pulmonary alveolar type 2 epithelial primary cells. FIG. 3A depicts green fluorescence stained SMS cells co-cultured with adherent human alveolar type 2 epithelial cells (image at 100×). FIG. 3B depicts flow cytometric detection of green fluorescence in human pulmonary alveolar type 2 epithelial primary cells with atached fluroescence-stained SMS cells. Alveolar epithelial cells incubated with SMS cells resulted in 90% live cells counted, while control alveolar epithelial cells resulted in 93% live cells counted. FIG. 3C depicts the total number of cells for both treated (with SMS cells) and non-treated pulmonary alveolar type 2 epithelial primary cells. Two replicates were performed.

### DETAILED DESCRIPTION

Embodiments provided herein relate to methods and compositions to treat, ameliorate, inhibit, prevent, or delay viral, fungal, or bacterial infections or the damage resulting from a viral, fungal, or bacterial infection, such as a coronavirus infection or a pneumonia infection, or an inflammatory disease, including but not limited to inflammatory diseases resulting from a viral, fungal, or bacterial infection. The methods and compositions repair or regenerate tissue or organs damaged by, e.g., the viral, fungal, or bacterial infection, or a sequela thereof, or inflammatory disease, thereby providing an improved treatment or therapy for the viral, fungal, or bacterial infection, or the sequela thereof, or inflammatory disease.

In some embodiments, the methods comprise administering to a subject or patient having a viral, fungal, or bacterial infection, such as a coronavirus infection, or a sequela thereof, a therapeutically effective amount of a composition comprising small mobile stem (SMS) cells, alone or in combination with a therapeutically effective amount of an appropriate antibiotic, antifungal, or anti-viral agent, such as azithromycin, a vaccine or remdesivir or any combination thereof. Said subject can be selected or identified to receive a stem cell therapy and such selection or identification can be made by diagnostic and/or clinical evaluation confirming the presence of a viral infection, such as MERS-CoV, SARS-CoV, or SARS-CoV-2.

In some embodiments are also disclosed methods of treating or inhibiting an inflammatory disease, comprising selecting a subject having the inflammatory disease and administering to the subject a composition comprising a therapeutically effective amount of SMS cells, alone or in combination with a therapeutically effective amount of an appropriate standard care of treatment.

Also provided are compositions that comprise a therapeutically effective amount of SMS cells alone, or in combination with a therapeutically effective amount of a standard of care treatment, such as an appropriate antibiotic, antifungal, anti-viral agent, such as azithromycin, a vaccine or remdesivir or both.

### I. Definitions

All patents, applications, published applications and other publications referenced herein are expressly incorporated by reference in their entireties unless stated otherwise. In the event that there are a plurality of definitions for a term herein, those in this section prevail unless stated otherwise.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of ” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In some embodiments, the “purity” of any given agent (e.g., antibody, polypeptide binding agent) in a composition may be specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

As used herein, the terms “function” and “functional” and the like refer to a biological, enzymatic, or therapeutic function.

The term “isolated” is meant material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

The practice of the present disclosure will employ, unless indicated specifically to the contrary, conventional methods of molecular biology and recombinant DNA techniques within the skill of the art, many of which are described below for the purpose of illustration. Such techniques are explained fully in the literature. See, e.g. , Sambrook, el al, Molecular Cloning: A Laboratory Manual (3rd Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5^{th} Ed. Hoboken N.J., John Wiley & Sons; B. Perbal, A Practical Guide to Molecular Cloning (3^{rd} Edition 2010); Farrell, R., RNA Methodologies: A Laboratory Guide for Isolation and Characterization (3^{rd} Edition 2005).

As used herein, a “subject”, “individual”, or “patient” refers to an animal that is the object of therapy, treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “treating,” “improving,” “promoting,” or “therapy,” do not necessarily mean total cure or abolition of damaged tissue or of a viral infection. Any alleviation of any undesired signs or symptoms of a viral, fungal, or bacterial infection or of improvement in the repair, growth, or regeneration of damaged tissue, to any extent can be considered treatment and/or therapy. Treatment may include enhancing, improving, accelerating, promoting, or ameliorating tissue damage or regeneration.

The term “inhibit” as used herein refers to the reduction or prevention of a viral, fungal, or bacterial infection or the damage caused by such an infection, such as SARS-CoV-2. The reduction can be by 10%, 20%, 30%, 40%, 50%, 60%, 70%, 80%, 90%, or 100%, or an amount that is within a range defined by any two of the aforementioned values. As used herein, the term “delay” has its ordinary meaning as understood in light of the specification, and refers to a slowing, postponement, or deferment of an event, such as a viral infection, to a time which is later than would otherwise be expected. The delay can be a delay of 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “viral infection” has its ordinary meaning as understood in light of the specification, and refers to an infection of a subject by a virus. As used herein, the term “virus” has its ordinary meaning as understood in light of the specification, and refers to obligate intracellular parasites of living but noncellular nature, consisting of DNA or RNA and a protein coat. Viruses range in diameter from about 20 to about 300 nm. Class I viruses (Baltimore classification) have a double-stranded DNA as their genome (such as Adenoviruses, Herpesviruses, or Poxviruses); Class II viruses have a single-stranded DNA as their genome (such as Parvoviruses); Class III viruses have a double-stranded RNA as their genome (such as Reoviruses); Class IV viruses have a positive single-stranded RNA as their genome, the genome itself acting as mRNA (such as Picornaviruses or Togaviruses); Class V viruses have a negative single-stranded RNA as their genome used as a template for mRNA synthesis (such as Orthomyxoviruses or Rhabdoviruses); Class VI viruses have a positive single-stranded RNA genome but with a DNA intermediate not only in replication but also in mRNA synthesis (such as Retroviruses); and Class VII viruses have a double-stranded DNA genome but with an RNA intermediate in life-cycle (such as Hepadnaviruses). The majority of viruses are recognized by the diseases they cause in plants, animals and prokaryotes. Viruses of prokaryotes are known as bacteriophages.

In some embodiments, the virus is a DNA virus. DNA viruses include, but are not limited to a virus belonging to one of the following families: adenovirus, astrovirus, hepadnavirus, herpesvirus, papovavirus, or poxvirus. In other embodiments, the virus is an RNA virus. RNA viruses include but are not limited to a virus belonging to one the following families: arenavirus, bunyavirus, calicivirus, coronavirus, Filovirus, flavivirus, orthomyxovirus, paramyxovirus, picornavirus, reovirus, retrovirus, rhabdovirus, or togavirus.

In some embodiments, the methods and compositions described herein are directed to a non-human animal. In some embodiments, the non-human animal virus may be selected from a picornavirus, such as a bovine enterovirus, a porcine enterovirus B, a foot-and-mouth disease virus, an equine rhinitis A virus, a bovine rhinitis B virus, a ljungan virus, equine rhinitis B virus, an aichi virus, a bovine kobuvirus, a porcine teschovirus, a porcine sapelovirus, a simian sapelovirus, an avian sapelovirus, an avian encephalomyelitis virus, a duck hepatitis A virus, or a simian enterovirus A; a pestivirus, such as border disease virus, a bovine virus diarrhea, or a classical swine fever virus; an arterivirus, such as an equine arteritis virus, a porcine reproductive and respiratory syndrome virus, a lactate dehydrogenase elevating virus, or a simian haemorrhagic fever virus; a coronavirus, such as a bovine coronavirus, a porcine coronavirus, a feline coronavirus, or a canine coronavirus; a paramyxovirus, such as a hendra virus, a nipah virus, a canine distemper virus, a rinderpest virus, a Newcastle disease virus, or a bovine respiratory syncytial virus; an orthomyxovirus, such as an influenza A virus, an influenza B virus, or an influenza C virus; a reovirus, such as a bluetongue virus; a porcine circovirus, a herpesvirus, such as a pseudorabies virus or a bovine herpesvirus 1; an asfarvirus, such as an African swine fever virus; a retrovirus, such as a simian immunodeficiency virus, a feline immunodeficiency virus, a bovine immunodeficiency virus, a bovine leukemia virus, a feline leukemia virus, a Jaagsiekte sheep retrovirus, or a caprine arthritis encephalitis virus; a flavivirus, such as a yellow fever virus, a West Nile virus, a dengue fever virus, a tick borne encephalitis virus, or a bovine viral diarrhea; or a rhabdovirus, such as a rabies virus.

In some embodiments, the methods and compositions described herein are directed to a human subject. In some embodiments, the human subject virus may be selected from a coronavirus, an adenovirus, an astrovirus, a hepadnavirus, a herpesvirus, a papovavirus, a poxvirus, an arenavirus, a bunyavirus, a calicivirus, a filovirus, a flavivirus, an orthomyxovirus, a paramyxovirus, a picornavirus, a reovirus, a retrovirus, a rhabdovirus, or a togavirus.

In some embodiments, the coronavirus includes, but is not limited to, a human coronavirus (etiologic agent of severe acute respiratory syndrome (SARS)), such as SARS-COV-2 (OR 2019-NCOV). In some embodiments, the adenovirus includes, but is not limited to, a human adenovirus. In some embodiments, the astrovirus includes, but is not limited to, a mamastrovirus. In some embodiments, the hepadnavirus includes, but is not limited to, the hepatitis B virus. In some embodiments, the herpesvirus includes, but is not limited to, a herpes simplex virus type I, a herpes simplex virus type 2, a human cytomegalovirus, an Epstein-Barr virus, a varicella zoster virus, a roseolovirus, or a Kaposi's sarcoma-associated herpesvirus. In some embodiments, the papovavirus includes, but is not limited to, human papilloma virus or a human polyoma virus. In some embodiments, the poxvirus includes, but is not limited to, a variola virus, a vaccinia virus, a cowpox virus, a monkeypox virus, a smallpox virus, a pseudocowpox virus, a papular stomatitis virus, a tanapox virus, a yaba monkey tumor virus, or a molluscum contagiosum virus. In some embodiments, the arenavirus includes, but is not limited to lymphocytic choriomeningitis virus, a lassa virus, a machupo virus, or a junin virus. In some embodiments, the bunyavirus includes, but is not limited to, a hanta virus, a nairovirus, an orthobunyavirus, or a phlebovirus. In some embodiments, the calicivirus includes, but is not limited to, a vesivirus, a norovirus, such as the Norwalk virus or a sapovirus. In some embodiments, the filovirus includes, but is not limited to, an Ebola virus or a Marburg virus. In some embodiments, the flavivirus includes, but is not limited to, a yellow fever virus, a West Nile virus, a dengue fever virus, a hepatitis C virus, a tick borne encephalitis virus, a Japanese encephalitis virus, a Murray Valley encephalitis virus, a St. Louis encephalitis virus, a Russian spring-summer encephalitis virus, a Omsk hemorrhagic fever virus, a bovine viral diarrhea virus, a Kyasanus Forest disease virus, or a Powassan encephalitis virus. In some embodiments, the orthomyxovirus includes, but is not limited to, influenza virus type A, influenza virus type B, or influenza virus type C. In some embodiments, the paramyxovirus includes, but is not limited to, a parainfluenza virus, a rubula virus (mumps), a morbillivirus (measles), a pneumovirus, such as a human respiratory syncytial virus, or a subacute sclerosing panencephalitis virus. In some embodiments, the picornavirus includes, but is not limited to, a poliovirus, a rhinovirus, a coxsackievirus A, a coxsackievirus B, a hepatitis A virus, an echovirus, or an enterovirus. In some embodiments, the reovirus includes, but is not limited to, a Colorado tick fever virus or a rotavirus. In some embodiments, the retrovirus includes, but is not limited to, a lentivirus, such as a human immunodeficiency virus, or a human T-lymphotrophic virus (HTLV). In some embodiments, the rhabdovirus includes, but is not limited to, a lyssavirus, such as the rabies virus, the vesicular stomatitis virus or the infectious hematopoietic necrosis virus. In some embodiments, the togavirus includes, but is not limited to, an alphavirus, such as a Ross river virus, an O'nyong'nyong virus, a Sindbis virus, a Venezuelan equine encephalitis virus, an Eastern equine encephalitis virus, a Western equine encephalitis virus, or a rubella virus.

Coronaviruses are enveloped positive-stranded RNA viruses that belong to the family Coronaviridae and the order Nidovirales. They possess RNA that is translated directly into one or more polyproteins, which are subsequently cleaved by virus proteases into mature or intermediate viral proteins. Those viral proteases are indispensable for virus replication. Coronaviruses typically result in respiratory and enteric infections affecting both animals and humans. They are considered relatively benign to humans: people around the globe are frequently infected with four human coronaviruses (229E, NL63, 0C43, and HKU1) typically leading to an upper respiratory tract infection manifested by common cold symptoms. However, coronaviruses can evolve into a strain that can infect human subjects leading to fatal illness. Examples are SARS-CoV, MERS-CoV, and the recently identified SARS-CoV-2 (or 2019-nCoV).

SARS-CoV-2 is highly contagious and has caused coronavirus disease 2019 (COVID-19) outbreaks worldwide. Its infection constitutes an important public health problem, as well as a potential bioterrorism threat. The problem is further exacerbated by the limited number of specific anti-SARS-CoV-2 therapeutics or vaccines. Therefore, there is currently an urgent and unmet need for the development of antiviral therapeutics for the treatment and prevention of SARS-CoV-2 infection and the amelioration of tissue damage caused by this virus.

Viral infections, including SARS-CoV-2, disrupt tissue homeostasis by altering cell function and architecture. In response to tissue damage, organ systems initiate an inflammatory response, encompassing combined effects of vascular changes and cellular reactions to initiate healing process. Viral infections can elicit acute and/or chronic inflammatory responses, resulting in short-term and/or long-term tissue damage. For example, in SARS-CoV-2, the pathophysiology of severe respiratory syndrome is multifactorial, manifesting with hyper-immune response and concomitant inflammatory cytokine storm (associated with other end organ impairment), lysis of alveolar and bronchial epithelial cells, and often secondary opportunistic pneumonia. In the absence of adequate therapy, such as in COVID-19, the tissue damage is chronic. Thus, even after the underlying viral infection has passed, tissue damage remains a concern.

Stem cells are immature, unspecialized cells capable of renewing themselves for extended periods through cell division. Under certain conditions, they differentiate into mature, functional cells. Stem cells play various roles in tissue repair and regeneration. The use of stem cells and stem cell derivatives is currently of great interest to medical research, particularly for the prospects of providing reagents for treating tissue which has been damaged by various causes such as genetic disorders, injuries, or disease.

Small mobile stem (SMS) cells have recently been isolated and characterized, for example in WO 2014/200940 and WO 2017/172638, each of which is hereby expressly incorporated by reference in its entirety. SMS cells are adherent cells that are from 4.5 to 5.5 μm in diameter, and are obtained from sources such as umbilical cord, peripheral blood, bone marrow, or solid tissue. In some embodiments, SMS cells are used to repair or regenerate damaged tissue as a result of a viral infection.

SMS cells exhibit exceptional features conducive to tissue repair and regeneration. SMS cells are distinguished from other stem cell types, such as mesenchymal stem cells (MSCs), in that they can be isolated easily from plasma, they are much smaller, difficult to stain for microscopy, and are not significantly immunogenic.

Although human bone marrow MSCs have been safely administered in some patients with acute respiratory distress syndrome (ARDS) and septic shock (phase I/II trials), there are still substantial short and long term safety concerns with intravenous administration of MSCs. The infusion of MSCs may result in agglutination (clumping) of these relatively large cells within injured capillary beds, thereby contributing to local tissue ischemia and infarction, and therefore parenchymal injury. Beyond that, MSCs may be sufficiently immunogenic that they may be targeted by the host immune system.

These concerns are minimized when providing infected subjects with a therapy that includes SMS cells, which have relatively few membrane proteins, and are thus not prone to adhere to each other to form clumps (as do MSCs). Thus, SMS cells are safer for therapeutic intravenous infusion compared to MSCs. In addition, SMS cells have few immunogenic membrane proteins, and are found in the circulatory system, and thus concerns regarding intra-circulatory administration or graft rejection are minimal.

SMS cells exhibit a profound effect on tissue regeneration in vitro and in cell culture, these cells have demonstrated a robust angiogenic effect. Accordingly, embodiments provided herein relate to compositions comprising SMS cells and methods of using these compositions comprising administering SMS cells to a subject with a viral, fungal, or bacterial infection to mitigate parenchymal injury and/or repair pulmonary damage wrought by such pathogens including SARS-CoV-2, optionally selecting said subject, preferably a human, to receive a therapy that improves pulmonary recovery after said viral, fungal, or bacterial infection. For instance, said subject can be selected or identified by conventional diagnostic methods or clinical evaluation of symptoms that accompany SARS-CoV-2 infection.

Experimental animal studies have demonstrated that SMS cells can bring about wound healing in injured skin, especially promoting angiogenesis and granulation. Thus, SMS cells are used herein to mitigate or ameliorate the damage wrought by the intense inflammatory state of SARS-CoV-2 related cytokine storm, reduce or inhibit alveolar and/or respiratory endothelial cell injury, and facilitate a more rapid repair of damaged lung tissue. SMS cells were found to reduce fibroblast activity and can be administered to subjects in need thereof so as to decrease the development of pulmonary fibrosis after viral, fungal, or bacterial infection. Taken together, the SMS cells described herein are used for the treating, mitigating, ameliorating, reducing, or shortening the course of critical illness from viral, fungal, or bacterial infections such as COVID-19, specifically SARS-CoV-2, reducing the overall mortality from the infection, and/or reducing the post-infectious debility in recovered patients.

As used herein, the term “mesenchymal stem cell (MSC)” refers to rare fibroblast-like cells capable of differentiating into a variety of cell types including bone, cartilage, and fat cells. Conventional surface markers used to define human MSCs are CD73, CD90, and CD105 positive, and CD14, CD19, CD34, CD45, and HLA-DR negative. Mesenchymal stem cells are known to have immunomodulatory properties on various immune cells through factors such as prostaglandin E2, nitric oxide, indoleamine 2,3-dioxygenase, IL-6, FasL, PD-L1, and PD-L2. Populations of mesenchymal stem cells can be isolated from several sources including adipose tissue, dental pulp, peripheral blood, or birth-derived tissues such as amniotic fluid, umbilical cord, or placenta.

As used herein, the term “fibroblast” refers to common cells that make up connective tissue. These cells are able to synthesize extracellular matrix and collagen, forming the stroma in biological tissues. Fibroblasts are also involved in inflammation during tissue injury and/or pathogenic invasion through chemokine signaling. Fibrosis, the process of tissue remodeling and formation of scar tissue that replaces normal parenchymal tissue, is caused by stimulated fibroblasts producing new connective tissue. Fibrosis can interfere with normal organ function and is a common sequela of chronic or acute inflammation.

As used herein, the term “pulmonary alveolar epithelial cells” refers to the population of cells comprised of type I and type II alveolar epithelial cells lining the majority of the lung surface. Type I alveolar cells (over 95% of the population) are large squamous cells involve in the process of gas exchange between the alveoli and the blood. Type II alveolar cells (between 2-5% of the population) produce pulmonary surfactant. While type I alveolar epithelial cells are generally unable to replicate, type II alveolar epithelial cells can proliferate and act as progenitor cells to other cell types, including type I alveolar epithelial cells.

In some embodiments, the compositions and methods provided may be used for prevention or inhibition of tissue damage. For example, the compositions comprising SMS cells may be used on a subject who does not have a viral, fungal, or bacterial infection for the purpose of strengthening or bolstering tissues or cells that are not damaged, such that if a viral, fungal, or bacterial infection were to occur, the damage to the tissue would be prevented, minimized, or insubstantial due to the preventative measures.

Also provided herein are compositions and methods for promoting or improving repair or regenerations of damaged tissue, or for improving recovering from a viral, fungal, or bacterial infection, wherein the composition provided to the subject in need comprises a therapeutically effective amount of SMS cells.

As disclosed herein, SMS cells are found to modulate gene expression in human cells. In some embodiments, Dickkopf-related protein 1 (DKK1), N-acylglucosamine 2-epimerase (RENBP), growth/differentiation factor 15 (GDF15), dermokine (DMKN), ferritin, light (FTL), tryptophan 2,3-dioxygenase (TDO2), apolipoprotein E (APOE), chitinase-3-like protein 1 (CHI3L1), or nuclear receptor subfamily 4 group A member 2 (NR4A2), or any combination thereof, are unregulated in human cells when contacted with SMS cells or compositions comprising the SMS cells. In some embodiments, interleukin-11 (IL-11), SPRY domain-containing SOCS box protein 1 (SPSB1), cytochrome P450 26B1 (CYP26B1), or frizzled-8 (FZD8), or any combination thereof, is downregulated in human cells when contacted with SMS cells or compositions comprising the SMS cells. In some embodiments, modulation of gene expression in cells have effects on cell signaling, blood vessel diameter, cell differentiation, cell proliferation, or pathogen protection or any combination thereof. In some embodiments, the human cells contacted with the SMS cells in these embodiments are pulmonary alveolar type 2 cells.

As conventionally understood, Dickkopf-related protein (DKK1) is a major Wnt antagonist contributing to the suppression of Wnt/β-catenin signaling in alveolar epithelial cells during acute lung inflammation. Intratracheal administration of Wnt3a or an anti-DKK1 antibody has been shown to inhibit neutrophil influx into the alveolar airspace of injured lungs. In some embodiments, DKK1 expression in cells, such as pulmonary alveolar type 2 cells, is activated or increased by contact with SMS cells. In some embodiments, DKK1 expression in cells, such as pulmonary alveolar type 2 cells, is activated or increased by contact with SMS cells e.g., by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, or any percentage within a range defined by any two of the aforementioned percentages. In some embodiments, DKK1 expression in cells, such as pulmonary alveolar type 2 cells, is activated or increased by contact with SMS cells by 55%, about 55%, at least 55%, or at least about 55%. Accordingly, some embodiments concern methods to increase DKK1 expression in cells, such as pulmonary alveolar type 2 cells, by contacting said cells, such as pulmonary alveolar type 2 cells, with SMS cells and, optionally evaluating or measuring the DKK1 expression in said cells, such as pulmonary alveolar type 2 cells, after contact with said SMS cells and/or, optionally, selecting a subject to receive a medicament that increases DKK1 expression and administering to said subject SMS cells, preferably by contacting cells of said subject, such as pulmonary alveolar type 2 cells, with said SMS cells.

As conventionally understood, N-acylglucosamine 2-epimerase (RENBP) inhibits renin, lowers blood pressure and causes an increase in blood vessel diameter (vasodilation). In some embodiments, RENBP expression in cells, such as pulmonary alveolar type 2 cells, is activated or increased by contact with SMS cells. In some embodiments, RENBP expression in cells, such as pulmonary alveolar type 2 cells, is activated or increased by contact with SMS cells e.g., by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, or any percentage within a range defined by any two of the aforementioned percentages. In some embodiments, RENBP expression in cells, such as pulmonary alveolar type 2 cells, is activated or increased by contact with SMS cells by 60%, about 60%, at least 60%, or at least about 60%. Accordingly, some embodiments concern methods to increase RENBP expression in cells, such as pulmonary alveolar type 2 cells, by contacting said cells, such as pulmonary alveolar type 2 cells, with SMS cells and, optionally evaluating or measuring the RENBP expression in said cells, such as pulmonary alveolar type 2 cells, after contact with said SMS cells and/or, optionally, selecting a subject to receive a medicament that increases RENBP expression and administering to said subject SMS cells, preferably by contacting cells of said subject, such as pulmonary alveolar type 2 cells, with said SMS cells.

As conventionally understood, growth/differentiation factor 15 (GDF15) and dermokine (DMKN) both cause epithelial to mesenchymal transition (EMT). In some embodiments, GDF15 and/or DMKN expression in cells, such as pulmonary alveolar type 2 cells, is activated or increased by contact with SMS cells. In some embodiments, GDF15 expression in cells, such as pulmonary alveolar type 2 cells, is activated or increased by contact with SMS cells e.g., by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, or any percentage within a range defined by any two of the aforementioned percentages. In some embodiments, GDF15 expression in cells, such as pulmonary alveolar type 2 cells, is activated or increased by contact with SMS cells by 58%, about 58%, at least 58%, or at least about 58%. In some embodiments, DMKN expression in cells, such as pulmonary alveolar type 2 cells, is activated or increased by contact with SMS cells e.g., by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, or any percentage within a range defined by any two of the aforementioned percentages. In some embodiments, DMKN expression in cells, such as pulmonary alveolar type 2 cells, is activated or increased by contact with SMS cells by 130%, about 130%, at least 130%, or at least about 130%. Accordingly, some embodiments concern methods to increase GDF15 and/or DMKN expression in cells, such as pulmonary alveolar type 2 cells, by contacting said cells, such as pulmonary alveolar type 2 cells, with SMS cells and, optionally evaluating or measuring the GDF15 and/or DMKN expression in said cells, such as pulmonary alveolar type 2 cells, after contact with said SMS cells and/or, optionally, selecting a subject to receive a medicament that increases GDF15 and/or DMKN expression and administering to said subject SMS cells, preferably by contacting cells of said subject, such as pulmonary alveolar type 2 cells, with said SMS cells.

As conventionally understood, ferritin, such as ferritin, light (FTL) and ferritin, heavy (FTH), have been associated with pathogen protection of the lung and angiogenesis. In some embodiments, FTL and/or FTH expression in cells, such as pulmonary alveolar type 2 cells, is activated or increased by contact with SMS cells. In some embodiments, FTL and/or FTH expression in cells, such as pulmonary alveolar type 2 cells, is activated or increased by contact with SMS cells e.g., by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, or any percentage within a range defined by any two of the aforementioned percentages. In some embodiments, FTL and/or FTH expression in cells, such as pulmonary alveolar type 2 cells, is activated or increased by contact with SMS cells by 70%, about 70%, at least 70%, or at least about 70%. Accordingly, some embodiments concern methods to increase FTL and/or FTH expression in cells, such as pulmonary alveolar type 2 cells, by contacting said cells, such as pulmonary alveolar type 2 cells, with SMS cells and, optionally evaluating or measuring the FTL and/or FTH expression in said cells, such as pulmonary alveolar type 2 cells, after contact with said SMS cells and/or, optionally, selecting a subject to receive a medicament that increases FTL and/or FTH expression and administering to said subject SMS cells, preferably by contacting cells of said subject, such as pulmonary alveolar type 2 cells, with said SMS cells.

As conventionally understood, tryptophan 2,3-dioxygenase (TDO2) is involved in pathogen protection of the lung. Activity of TDO2 depletes levels of tryptophan, which is essential for pathogens. Some pathogens susceptible to tryptophan deficiency include Streptococcus and viruses such as herpes simplex and measles. In some embodiments, TDO2 expression in cells, such as pulmonary alveolar type 2 cells, is activated or increased by contact with SMS cells. In some embodiments, TDO2 expression in cells, such as pulmonary alveolar type 2 cells, is activated or increased by contact with SMS cells e.g., by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, or any percentage within a range defined by any two of the aforementioned percentages. In some embodiments, TDO2 expression in cells, such as pulmonary alveolar type 2 cells, is activated or increased by contact with SMS cells by 85%, about 85%, at least 85%, or at least about 85%. Accordingly, some embodiments concern methods to increase TDO2 expression in cells, such as pulmonary alveolar type 2 cells, by contacting said cells, such as pulmonary alveolar type 2 cells, with SMS cells and, optionally evaluating or measuring the TDO2 expression in said cells, such as pulmonary alveolar type 2 cells, after contact with said SMS cells and/or, optionally, selecting a subject to receive a medicament that increases TDO2 expression and administering to said subject SMS cells, preferably by contacting cells of said subject, such as pulmonary alveolar type 2 cells, with said SMS cells.

As conventionally understood, apolipoprotein E (APOE) is involved in pathogen protection of the lung. APOE has been found to have anti-inflammatory and antioxidant effects that decrease severity of lung disease in murine models. In some embodiments, APOE expression in cells, such as pulmonary alveolar type 2 cells, is activated or increased by contact with SMS cells. In some embodiments, APOE expression in cells, such as pulmonary alveolar type 2 cells, is activated or increased by contact with SMS cells e.g., by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, or any percentage within a range defined by any two of the aforementioned percentages. In some embodiments, APOE expression in cells, such as pulmonary alveolar type 2 cells, is activated or increased by contact with SMS cells by 110%, about 110%, at least 110%, or at least about 110%. Accordingly, some embodiments concern methods to increase APOE expression in cells, such as pulmonary alveolar type 2 cells, by contacting said cells, such as pulmonary alveolar type 2 cells, with SMS cells and, optionally evaluating or measuring the APOE expression in said cells, such as pulmonary alveolar type 2 cells, after contact with said SMS cells and/or, optionally, selecting a subject to receive a medicament that increases APOE expression and administering to said subject SMS cells, preferably by contacting cells of said subject, such as pulmonary alveolar type 2 cells, with said SMS cells.

As conventionally understood, chitinase-3-like protein 1 (CHI3L1) is involved in pathogen protection of the lung. While still not fully understood, CHI3L1 has been associated with cytotoxicity against bacterial pathogens and modulation of cell death, inflammation, and cellular remodeling. In some embodiments, CHI3L1 expression in cells, such as pulmonary alveolar type 2 cells, is activated or increased by contact with SMS cells. In some embodiments, CHI3L1 expression in cells, such as pulmonary alveolar type 2 cells, is activated or increased by contact with SMS cells e.g., by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, or any percentage within a range defined by any two of the aforementioned percentages. In some embodiments, CHI3L1 expression in cells, such as pulmonary alveolar type 2 cells, is activated or increased by contact with SMS cells by 150%, about 150%, at least 150%, or at least about 150%. Accordingly, some embodiments concern methods to increase CHI3L1 expression in cells, such as pulmonary alveolar type 2 cells, by contacting said cells, such as pulmonary alveolar type 2 cells, with SMS cells and, optionally evaluating or measuring the CHI3L1 expression in said cells, such as pulmonary alveolar type 2 cells, after contact with said SMS cells and/or, optionally, selecting a subject to receive a medicament that increases CHI3L1 expression and administering to said subject SMS cells, preferably by contacting cells of said subject, such as pulmonary alveolar type 2 cells, with said SMS cells.

As conventionally understood, nuclear receptor subfamily 4 group A member 2 (NR4A2) acts as an inhibitor of inflammation and enhances DNA repair. In some embodiments, NR4A2 expression in cells, such as pulmonary alveolar type 2 cells, is activated or increased by contact with SMS cells. In some embodiments, NR4A2 expression in cells, such as pulmonary alveolar type 2 cells, is activated or increased by contact with SMS cells e.g., by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, or any percentage within a range defined by any two of the aforementioned percentages. In some embodiments, NR4A2 expression in cells, such as pulmonary alveolar type 2 cells, is activated or increased by contact with SMS cells by 55%, about 55%, at least 55%, or at least about 55%. Accordingly, some embodiments concern methods to increase NR4A2 expression in cells, such as pulmonary alveolar type 2 cells, by contacting said cells, such as pulmonary alveolar type 2 cells, with SMS cells and, optionally evaluating or measuring the NR4A2 expression in said cells, such as pulmonary alveolar type 2 cells, after contact with said SMS cells and/or, optionally, selecting a subject to receive a medicament that increases NR4A2 expression and administering to said subject SMS cells, preferably by contacting cells of said subject, such as pulmonary alveolar type 2 cells, with said SMS cells.

As conventionally understood, interleukin-11 (IL-11) is a major cytokine associated with fibrosis, parenchymal dysfunction, and chronic inflammation of the airway. In some embodiments, IL-11 expression in cells, such as pulmonary alveolar type 2 cells, is inhibited or reduced by contact with SMS cells. In some embodiments, IL- 11 expression in cells, such as pulmonary alveolar type 2 cells, is inhibited or reduced by contact with SMS cells e.g., by at least 10%, 20%, 30%, 40%, 50%, 60%, 70%, 80%, 90%, or 100%, or any percentage within a range defined by any two of the aforementioned percentage. In some embodiments, IL-11 expression in cells, such as pulmonary alveolar type 2 cells, is inhibited or reduced by contact with SMS cells by 40%, about 40%, at least 40%, or at least about 40%. Accordingly, some embodiments concern methods to inhibit or reduce IL-11 expression in cells, such as pulmonary alveolar type 2 cells, by contacting said cells, such as pulmonary alveolar type 2 cells, with SMS cells and, optionally evaluating or measuring the IL-11 expression in said cells, such as pulmonary alveolar type 2 cells, after contact with said SMS cells and/or, optionally, selecting a subject to receive a medicament that reduces or inhibits IL-11 expression and administering to said subject SMS cells, preferably by contacting cells of said subject, such as pulmonary alveolar type 2 cells, with said SMS cells.

As conventionally understood, SPRY domain-containing SOCS box protein 1 (SPSB1) negatively regulates nitric oxide (NO) and causes vessel diameter reduction (vasoconstriction). In some embodiments, SPSB1 expression in cells, such as pulmonary alveolar type 2 cells, is inhibited or reduced by contact with SMS cells. In some embodiments, SPSB1 expression in cells, such as pulmonary alveolar type 2 cells, is inhibited or reduced by contact with SMS cells e.g., by at least 10%, 20%, 30%, 40%, 50%, 60%, 70%, 80%, 90%, or 100%, or any percentage within a range defined by any two of the aforementioned percentage. In some embodiments, SPSB1 expression in cells, such as pulmonary alveolar type 2 cells, is inhibited or reduced by contact with SMS cells by 50%, about 50%, at least 50%, or at least about 50%. Accordingly, some embodiments concern methods to inhibit or reduce SPSB1 expression in cells, such as pulmonary alveolar type 2 cells, by contacting said cells, such as pulmonary alveolar type 2 cells, with SMS cells and, optionally evaluating or measuring the SPSB1 expression in said cells, such as pulmonary alveolar type 2 cells, after contact with said SMS cells and/or, optionally, selecting a subject to receive a medicament that reduces or inhibits SPSB1 expression and administering to said subject SMS cells, preferably by contacting cells of said subject, such as pulmonary alveolar type 2 cells, with said SMS cells.

As conventionally understood, cytochrome P450 26B1 (CYP26B1) catabolizes retinoic acid, which is essential for lung differentiation. In some embodiments, CYP26B1 expression in cells, such as pulmonary alveolar type 2 cells, is inhibited or reduced by contact with SMS cells. In some embodiments, CYP26B1 expression in cells, such as pulmonary alveolar type 2 cells, is inhibited or reduced by contact with SMS cells e.g., by at least 10%, 20%, 30%, 40%, 50%, 60%, 70%, 80%, 90%, or 100%, or any percentage within a range defined by any two of the aforementioned percentages. In some embodiments, CYP26B1 expression in cells, such as pulmonary alveolar type 2 cells, is inhibited or reduced by contact with SMS cells by 50%, about 50%, at least 50%, or at least about 50%. Accordingly, some embodiments concern methods to inhibit or reduce CYP26B1 expression in cells, such as pulmonary alveolar type 2 cells, by contacting said cells, such as pulmonary alveolar type 2 cells, with SMS cells and, optionally evaluating or measuring the CYP26B1 expression in said cells, such as pulmonary alveolar type 2 cells, after contact with said SMS cells and/or, optionally, selecting a subject to receive a medicament that reduces or inhibits CYP26B1 expression and administering to said subject SMS cells, preferably by contacting cells of said subject, such as pulmonary alveolar type 2 cells, with said SMS cells.

As conventionally understood, frizzled-8 (FZD8) is responsible for alveolar epithelial cell trans-differentiation, which is the process whereby type II alveolar epithelial cells differentiate to type I alveolar epithelial cells during lung recovery. In some embodiments, FZD8 expression in cells, such as pulmonary alveolar type 2 cells, is inhibited or reduced by contact with SMS cells. In some embodiments, FZD8 expression in cells, such as pulmonary alveolar type 2 cells, is inhibited or reduced by contact with SMS cells e.g., by at least 10%, 20%, 30%, 40%, 50%, 60%, 70%, 80%, 90%, or 100%, or any percentage within a range defined by any two of the aforementioned percentages. In some embodiments, FZD8 expression in cells, such as pulmonary alveolar type 2 cells, is inhibited or reduced by contact with SMS cells by 50%, about 50%, at least 50%, or at least about 50%. Accordingly, some embodiments concern methods to inhibit or reduce FZD8 expression in cells, such as pulmonary alveolar type 2 cells, by contacting said cells, such as pulmonary alveolar type 2 cells, with SMS cells and, optionally evaluating or measuring the FZD8 expression in said cells, such as pulmonary alveolar type 2 cells, after contact with said SMS cells and/or, optionally, selecting a subject to receive a medicament that reduces or inhibits FZD8 expression and administering to said subject SMS cells, preferably by contacting cells of said subject, such as pulmonary alveolar type 2 cells, with said SMS cells.

The term “therapeutically effective amount” is used to indicate an amount of an active compound, or pharmaceutical agent, that elicits the biological or medicinal response indicated. For example, a therapeutically effective amount of compound can be the amount needed to prevent, alleviate or ameliorate symptoms of disease or prolong the survival of the subject being administered the therapy. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

### II. Methods of Isolating SMS Cells

Some embodiments provided herein relate to methods of isolating SMS cells and methods of making the compositions provided herein. Methods of isolating SMS cells have been described previously, including in WO 2014/200940 and WO 2017/172638, each of which is hereby incorporated by reference herein in its entirety.

In some embodiments, the methods comprise culturing a population of SMS cells for a time sufficient to generate a therapeutically effective amount of SMS cells.

“Cell culture” or “cultured cell”, as used herein, refer to cells or tissues that are maintained, cultured, cultivated or grown in an artificial, in vitro environment. Included within this term are continuous cell lines (e.g. with an immortal phenotype), primary cell cultures, finite cell lines (e.g., non-transformed cells), and any other cell population maintained in vitro. In this connection, a primary cell is a cell that is directly obtained from a tissue or organ of an animal, including a human, in the absence of culture. Typically, though not necessarily, a primary cell is capable of undergoing ten or fewer passages in vitro before senescence or cessation of proliferation.

“Maintenance” means continued survival of a cell or population of cells, at times, with or without an increase in the numbers of cells. “Proliferation”, “propagation”, “expansion” and “growth”, which may be used interchangeably with each other, refer to an increase in cell number. According to one embodiment, this term refers to a continuous survival of the cells for at least 6, 7, 8, 9, 10, 11, 12, 13, 14, 15, 16, 17, 18, 19, 20, 21, 22, 23, 24, 25, 26, 32, 48, 52, 104 or more weeks or within a range defined by any two of the aforementioned time frames. In the alternative, the continuous survival of the cells continues for at least 25, 26, 27, 28, 29, or 30 or more passages or within a range defined by any two of the aforementioned number of passages.

“Cell suspension” as used herein, refers to a culture of cells in which the majority of the cells freely float in the medium, typically a culture medium (system), and the cells floating as single cells, as cell clusters and/or as cell aggregates. In other words, the cells survive and propagate in the medium without being attached to a solid or semi solid substrate. “Adherent cells” as used herein refers to a cell or cell population that adheres to a substrate or surface.

“Culture system” as used herein, refers to culture conditions for supporting the maintenance and propagation of SMS cells or somatic cells derived therefrom, as well as, under selected conditions, for supporting derivation and propagation of undifferentiated or differentiated SMS cells. The term denotes a combination of elements, which can include a basic medium (a cell culture medium usually including a defined base solution, which includes salts, sugars and amino acids) and a serum replacement supplement. The culture system may further include other elements such as, without being limited thereto, an extracellular matrix (ECM) component, additional serum or serum replacements, a culture (nutrient) medium and other exogenously added factors, which together provide suitable conditions that support SMS cell growth, cell culture maintenance, cell differentiation, or expression of various molecules. In the relevant context, the term “culture system” also encompasses the cells cultured therein.

SMS cells may be cultured in T25 flasks using growth medium (e.g., in an incubator at 37° C. and 5% CO₂). The SMS cell population may contain a heterogeneous cell population of undifferentiated SMS cells and SMS derived differentiated cells. SMS undifferentiated cells are present as a floating and an adherent fraction and the floating fraction is predominantly undifferentiated SMS cells (e.g., greater than or equal to 50%, 55%, 60%, 65%, 70%, 75%, 80%, 85%, 90%, or 95% undifferentiated SMS cells or an amount of undifferentiated SMS cells that is within a range defined by any two of the aforementioned values). Accordingly, some alternatives concern a suspension of non- adherent and undifferentiated SMS cells, which are preferably grown in a liquid media in a manner that prevents adherence (e.g., in a polypropylene vessel or flask). In some alternatives, the floating and undifferentiated SMS cell population is greater than or equal to 50%, 55%, 60%, 65%, 70%, 75%, 80%, 85%, 90%, or 95% undifferentiated SMS cells or an amount of undifferentiated SMS cells that is within a range defined by any two of the aforementioned values.

Undifferentiated SMS cells can be isolated or purified by differential centrifugation, removing clumps of cells or differentiated cells at low centrifugation speed followed by centrifuging undifferentiated SMS cells at high speed. Alternatively, the undifferentiated cells may be isolated by filtration, including differential filtration using filters having progressively smaller pore sizes to a pore size of 3-5 μm. Alternatively, the undifferentiated cells may be isolated by immune conjugation (e.g., a binding partner specific for a stem cell receptor on SMS cells, wherein the binding partner, such as an antibody, is conjugated to a bead, such as a magnetic bead or via FACS cell sorting), or differential filtration using filters having progressively smaller pore sizes to a pore size of 3-5 μm The isolated undifferentiated SMS cells are examined under the microscope for homogeneity. By passing the cells for at least 25 passages (e.g., 25, 26, 27, 28, 29, 30, 31, 32, 33, 34, 35, 36, 37, 38, 39, or 40 passages or within a range defined by any two of the aforementioned number of passages) prior to employing one or more of the isolation protocols above, one can obtain an improved homogeneous population of SMS cells.

The SMS cells can be grown a variety of mediums with or without serum and with or without inclusion of a differentiation induction compound (e.g., insulin). The cell growth medium is replaced every week by centrifuging the SMS cells at 4200g for 15 min. The centrifugation may be varied at 3000g, 3500g, 4000g, 4100g, 4200g, 4300g, 4500g, or 5000g or by centrifugation at a speed that is within a range defined by any two of the aforementioned speeds, and the time adjusted accordingly. Under these conditions, the volume size of the medium (cell crowdedness) is growth limiting to SMS cells. The SMS cell homogeneity is assessed microscopically and the SMS cell count is estimated by assessing spectroscopically turbidity of the suspension and/or by measuring the size of the pellet after centrifugation at high speed. Suspension cultures of SMS cells facilitate transfer and cloning as the cells are easily split and/or transferred to a new tube with growth medium and such methods of splitting and/or transferring a culture of cells from an existing culture to a new culture is performed without using an enzyme that liberates the cells from the culture dish or a basal cell layer (e.g., trypsin). SMS cells predominantly grow as individual cells, without clumping and the SMS cell suspensions can remain undifferentiated despite transfer procedures. The suspension culture is also scalable such that increasing the volume of the medium increases the number of cells obtained.

In some embodiments, the cultured SMS cells are used to formulate a compositions for administration to a subject. In some embodiments, the composition is formulated for various modes of administration, including for example, for topical, respiratory, or localized administration. In some embodiments, the methods of the present disclosure further contemplate systemic, enteral, or parenteral administration, such as respiratory (inhalation or intranasal), subcutaneous, intraperitoneal, intravenous, intramuscular, intraarterial, oral, enteric, subdermal, transdermal, sublingual, transbuccal, rectal, or vaginal administration.

In some embodiments, the cultured SMS cells are formulated for administration in combination with another therapy. In some embodiments, the therapy may be a standard of care therapy, such as a standard of care therapy for an inflammatory disease or a viral, fungal, or bacterial infection, as known in the art. For example, the SMS cells may be formulated for use in combination with an antibiotic, antifungal, or anti-viral therapy such as a vaccine or remdesivir.

As used herein, an “anti-viral” therapy including any method or agent used to treat, inhibit, manage, or ameliorate a viral infection. An anti-viral therapy can include, for example, a vaccine or an anti-viral agent, such as dexamethasone, convalescent serum, tocilizumab, sarilumab, ribavirin, favipiravir, darunavir, galidesivir, interferon alpha, interferon beta, lopinavir, ritonavir, remdesivir, triazavirin, umifenovir, or other agent or vaccine known or developed for the treatment, inhibition, or management of a viral infection.

### III. Method of Using The Compositions

Some embodiments provided herein relate to a method of promoting or improving repair or regeneration of damaged tissue or for improving recovery from a viral, fungal, or bacterial infection, or a sequela thereof, with a composition described herein. In some embodiments, the methods comprise selecting a subject suffering from a viral, fungal, or bacterial infection or a sequela thereof, and administering to the selected subject a composition comprising a therapeutically effective amount of small mobile stem (SMS) cells. In some embodiments, the SMS cells are obtained from peripheral blood. In some embodiments, the SMS cells are allogeneic to the subject. In some embodiments, the SMS cells are autologous to the subject. In some embodiments, the damaged tissue is lung tissue.

In some embodiments, promoting or improving repair or regeneration of damaged tissue or for improving recovery from a viral, fungal, or bacterial infection or a sequela thereof reduces alveolar cell injury, reduces respiratory endothelial cell injury, increases repair of damaged lung tissue, increases regeneration of damaged lung tissue, or reduces development of pulmonary fibrosis, or any combination thereof

In some embodiments, the damaged tissue is a result of the viral, fungal, or bacterial infection or a sequela thereof. In some embodiments, the viral infection is caused by a single-stranded RNA virus, a double-stranded RNA virus, a positive-sense ssRNA virus, a negative-sense ssRNA virus, a double-stranded DNA virus, or a single-stranded DNA virus. In some embodiments, the viral, fungal, or bacterial infection is caused by a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, filovirus, coxsackievirus, equine encephalitis virus, a Rift Valley fever virus, an alphavirus, hepatitis A virus, hepatitis B virus, hepatitis C virus, hepatitis D virus, or a hepatitis E virus, pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof. In some embodiments, the coronavirus is a MERS-CoV, SARS-CoV, or SARS-CoV-2. In some embodiments, the coronavirus is SARS-CoV-2. In some embodiments, the composition comprises aerosolized SMS cells. In some embodiments, the composition is administered via inhalation. In some embodiments, the composition is formulated for administration by inhalation. In some embodiments, the composition is administered intravenously. In some embodiments, the composition is formulated for intravenous administration.

A subject in need includes a subject that is suffering from a viral, fungal, or bacterial infection, such as from a SARS-CoV-2 infection, or is suspected of having a viral, fungal, or bacterial infection, or has recently recovered from a viral, fungal, or bacterial infection. A subject in need may have damaged tissue, such as damaged lung tissue, pulmonary fibrosis, or cell injury, or a combination thereof. A subject may be tested or diagnosed as having a viral, fungal, or bacterial infection, such as SARS-CoV-2 infection using real time reverse transcription polymerase chain reaction (RT-PCT), for example, or any other test for verification of infection, such as with antibody testing.

In some embodiments, the SMS cells are administered in combination with an antibiotic, antifungal, vaccine or an anti-viral medication. In some embodiments, the anti-viral medication is dexamethasone, convalescent serum, tocilizumab, sarilumab, ribavirin, favapiravir, darunavir, galidesivir, interferon alpha, interferon beta, lopinavir, ritonavir, remdesivir, triazavirin, umifenovir, or any combination thereof. In some embodiments, the vaccine is a coronavirus vaccine.

In some embodiments, administration of any one of the compositions disclosed herein to a subject upregulates Dickkopf-related protein 1 (DKK1), N-acylglucosamine 2-epimerase (RENBP), growth/differentiation factor 15 (GDF15), dermokine (DMKN), ferritin, light (FTL), tryptophan 2,3-dioxygenase (TDO2), apolipoprotein E (APOE), chitinase-3-like protein 1 (CHI3L1), or nuclear receptor subfamily 4 group A member 2 (NR4A2), or any combination thereof, in the subject. In some embodiments, administration of any one of the compositions disclosed herein to the subject downregulates interleukin-11 (IL-11), SPRY domain-containing SOCS box protein 1 (SPSB1), cytochrome P450 26B1 (CYP26B1), or frizzled-8 (FZD8), or any combination thereof, in the subject.

In some embodiments, the composition is administered to a subject in an amount and for a period sufficient to promote or improve repair or regeneration of damaged tissue or to improve recovery from a viral, fungal, or bacterial infection, or a sequela thereof. As can be appreciated, viral, fungal, or bacterial infections may vary significantly in severity, ability to heal, or chronicity, and therefore, the amount of composition or the length of time for treatment will vary. By way of example, the composition may be administered at least three times daily, once daily, once weekly, once monthly, or once yearly, or in a frequency within a range defined by any two of the aforementioned values.

Also disclosed herein are methods of treating or inhibiting an inflammatory disease. In some embodiments, the methods comprise selecting a subject having the inflammatory disease and administering to the subject a composition comprising a therapeutically effective amount of small mobile stem (SMS) cells. In some embodiments, the inflammatory disease is a pulmonary inflammatory disease. In some embodiments, the inflammatory disease comprises fibrosis. In some embodiments, the fibrosis is pulmonary fibrosis, liver fibrosis, kidney fibrosis, or cardiac fibrosis. In some embodiments, the inflammatory disease comprises chronic pulmonary obstructive disease (COPD). In some embodiments, the inflammatory disease comprises acute respiratory distress syndrome (ARDS). In some embodiments, the inflammatory disease is associated with a viral, fungal, or bacterial infection. In some embodiments, the viral infection is caused by a single-stranded RNA virus, a double-stranded RNA virus, a positive-sense ssRNA virus, a negative-sense ssRNA virus, a double-stranded DNA virus, or a single-stranded DNA virus. In some embodiments, the viral, fungal, or bacterial infection results from infection by a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, Filovirus, coxsackievirus, equine encephalitis virus, a Rift Valley fever virus, an alphavirus, hepatitis A virus, hepatitis B virus, hepatitis C virus, hepatitis D virus, or a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof. In some embodiments, the viral infection is a coronavirus infection. In some embodiments, the viral infection is a SARS-CoV, MERS-CoV, or SARS-CoV-2 infection. In some embodiments, the SMS cells are obtained from peripheral blood. In some embodiments, the SMS cells are allogeneic or autologous to the subject. In some embodiments, administering the composition reduces or inhibits alveolar cell injury, reduces or inhibits respiratory endothelial cell injury, increases or improves repair of damaged lung tissue, increases or enhances regeneration of damaged lung tissue, or reduces or inhibits the development of pulmonary fibrosis, or any combination thereof. In some embodiments, the composition is formulated for administration by inhalation e.g., via the nose or mouth. In some embodiments, the composition is formulated for intravenous administration. In some embodiments, the composition is administered with a standard of care treatment for the inflammatory disease. In some embodiments, the inflammatory disease is associated with a viral, fungal, or bacterial infection and the standard of care therapy is an antibiotic, antifungal, vaccine or an anti-viral medication, such as dexamethasone, convalescent serum, tocilizumab, sarilumab, ribavirin, favapiravir, darunavir, galidesivir, interferon alpha, interferon beta, lopinavir, ritonavir, remdesivir, triazavirin, umifenovir, or any combination thereof. In some embodiments, the vaccine is a vaccine against one or more of coronavirus, cholera, dengue, diphtheria, Haemophilus influenza type b infection, hepatitis A, hepatitis B, influenza, Japanese encephalitis, meningococcal meningitis, pertussis, polio, rabies, tetanus, tuberculosis, typhoid, or yellow fever. In some embodiments, the vaccine is a coronavirus COVID-19 vaccine.

In some embodiments, administration of any one of the compositions disclosed herein to the subject upregulates Dickkopf-related protein 1 (DKK1), N-acylglucosamine 2-epimerase (RENBP), growth/differentiation factor 15 (GDF15), dermokine (DMKN), ferritin, light (FTL), tryptophan 2,3-dioxygenase (TDO2), apolipoprotein E (APOE), chitinase-3-like protein 1 (CHI3L1), or nuclear receptor subfamily 4 group A member 2 (NR4A2), or any combination thereof, in the subject. In some embodiments, administration of any one of the compositions disclosed herein to the subject downregulates interleukin-11 (IL-11), SPRY domain-containing SOCS box protein 1 (SPSB1), cytochrome P450 26B1 (CYP26B1), or frizzled-8 (FZD8), or any combination thereof, in the subject.

### IV. Compositions

Some embodiments disclosed herein relate to compositions comprising a therapeutically effective amount of SMS cells for promoting or improving repair or regeneration of damaged tissue or for improving recovery from a viral infection.

“SMS cells” as used herein refers to a cell or a cell population characterized in that the cells are adherent cells of about 5 μm in diameter. The SMS cells are equi-dimensional, with strict radial symmetry, and exhibit a translucent cytoplasm and circular nucleus that includes a centrally located circle of a different light contrast, as viewed in a light microscope. In addition, SMS cells demonstrate an extraordinary resistance to various non-physiological conditions, including low and high temperature, freezing and thawing in standard growth medium, dehydration, high pH values, and variations of ionic strength. SMS cells are also characterized by their high mobility of up to about 1.5 μm/sec. In some embodiments, the SMS cells are obtained from peripheral blood.

The terms “effective amount” or “effective dose” is used to indicate an amount of an active compound, or pharmaceutical agent, that elicits the biological or medicinal response indicated. For example, an effective amount of compound can be the amount needed to alleviate or ameliorate symptoms of disease or prolong the survival of the subject being treated. This response may occur in a tissue, system, animal or human and includes alleviation of the signs or symptoms of the disease being addressed. Determination of an effective amount is well within the capability of those skilled in the art, in view of the disclosure provided herein. The effective amount of the compounds disclosed herein required as a dose will depend on the route of administration, the type of animal, including human, receiving therapy, and the physical characteristics of the specific animal under consideration. The dose can be tailored to achieve a desired effect, but will depend on such factors as weight, diet, concurrent medication and other factors which those skilled in the medical arts will recognize.

The term “pharmaceutically acceptable salts” includes relatively non-toxic, inorganic and organic acid, or base addition salts of compositions, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include phosphates, hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

“Formulation”, “pharmaceutical composition”, and “composition” as used interchangeably herein are equivalent terms referring to a composition of matter for administration to a subject.

The term “pharmaceutically acceptable” means compatible with therapy for a subject, and in particular, a human.

The terms “agent” refers to an active agent that has biological activity and may be used in a therapy. Also, an “agent” can be synonymous with “at least one agent,” “compound,” or “at least one compound,” and can refer to any form of the agent, such as a derivative, analog, salt or a prodrug thereof. The agent can be present in various forms, components of molecular complexes, and pharmaceutically acceptable salts (e.g., hydrochlorides, hydrobromides, sulfates, phosphates, nitrates, borates, acetates, maleates, tartrates, and salicylates). The term “agent” can also refer to any pharmaceutical molecules or compounds, therapeutic molecules or compounds, matrix forming molecules or compounds, polymers, synthetic molecules and compounds, natural molecules and compounds, and any combination thereof

In some embodiments, the compositions include a therapeutically effective amount of small mobile stem (SMS) cells for use in repairing or regenerating damaged tissue or for improving recovery from a viral, fungal, or bacterial infection, or a sequela thereof, in a subject in need thereof. In some embodiments, the viral, fungal, or bacterial infection is an infection from any bacteria, fungus, or virus, or any combination of bacteria, fungus, or viruses, including, for example, coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, filovirus, coxsackievirus, equine encephalitis virus, a Rift Valley fever virus, an alphavirus, hepatitis A virus, hepatitis B virus, hepatitis C virus, hepatitis D virus, or a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof. In some embodiments, the viral infection is an infection of MERS-CoV, SARS-CoV, or SARS-CoV-2. In some embodiments, the SMS cells are obtained from peripheral blood. In some embodiments, the SMS cells are obtained from a subject, where administration to the subject is autologous. In some embodiments, the SMS cell are obtained from an individual different from a subject, where administration to the subject is allogeneic. In some embodiments, the composition upregulates Dickkopf-related protein 1 (DKK1), N-acylglucosamine 2-epimerase (RENBP), growth/differentiation factor 15 (GDF15), dermokine (DMKN), ferritin, light (FTL), tryptophan 2,3-dioxygenase (TDO2), apolipoprotein E (APOE), chitinase-3-like protein 1 (CHI3L1), or nuclear receptor subfamily 4 group A member 2 (NR4A2), or any combination thereof, in the subject in need thereof. In some embodiments, the composition downregulates interleukin-11 (IL-11), SPRY domain-containing SOCS box protein 1 (SPSB1), cytochrome P450 26B1 (CYP26B1), or frizzled-8 (FZD8), or any combination thereof, in the subject in need thereof

In some embodiments, the compositions are formulated for suitable administration to a subject, including for example, for topical, respiratory, localized administration. In some embodiments, the methods of the present disclosure further contemplate systemic, enteral, or parenteral administration, such as inhalable, intranasal, subcutaneous, intraperitoneal, intravenous, intramuscular, intraarterial, oral, enteric, subdermal, transdermal, sublingual, transbuccal, rectal, or vaginal administration. In some embodiments, the composition is formulated for administration through a nebulizer, wherein the formulation is inhaled into the lungs or an inhaler, which can be used to introduce the composition into the nose, sinus, or throat.

In some embodiments, depending on the mode of administration, the composition is formulated as an aerosol, such as in an inhalant formulation suitable for an inhaler or for a nebulizer, or in an intranasal preparation. In some embodiments, the formulation is suitable in a pressurized metered-dose inhaler (pMDI), a dry powder inhaler (DPI), or a soft mist inhaler (SMI).

Also disclosed herein in some embodiments is the use of any one of the compositions disclosed herein for the treatment or inhibition of an inflammatory disease. In some embodiments, the inflammatory disease is pulmonary inflammatory disease. In some embodiments, the inflammatory disease comprises fibrosis. In some embodiments, the fibrosis is pulmonary fibrosis, liver fibrosis, kidney fibrosis, or cardiac fibrosis. In some embodiments, the inflammatory disease comprises chronic pulmonary obstructive disease (COPD). In some embodiments, the inflammatory disease comprises acute respiratory distress syndrome (ARDS). In some embodiments, the inflammatory disease is associated with a viral, fungal, or bacterial infection. In some embodiments, the viral infection is a coronavirus infection. In some embodiments, the viral infection is a SARS-CoV, MERS-CoV, or SARS-CoV-2 infection.

In some embodiments, the compositions further include a therapeutically effective amount of an at least one additional compound. In some embodiments, the at least one additional compound is an antibiotic such as azithromycin, an antifungal, such as Voriconazole, Amphotericin B or itraconazole, a vaccine or an anti-viral agent, such as dexamethasone, convalescent serum, tocilizumab, sarilumab, ribavirin, favapiravir, darunavir, galidesivir, interferon alpha, interferon beta, lopinavir, ritonavir, remdesivir, triazavirin, umifenovir, or other agent or vaccine known or developed for the treatment, inhibition, or management of a viral infection.

In some embodiments, the at least one addition compound is a pharmaceutically acceptable carrier, preservative, antioxidant, diluent, or excipient, or any combination thereof. In some embodiments, the at least one additional compound is a carrier. As used herein, a “carrier” refers to a compound that facilitates the incorporation of a compound into cells or tissues. Exemplary carriers include, but are not limited to, water, saline, buffered saline, dextrose, glycerol, ethanol, partial glyceride mixtures of saturated or unsaturated vegetable fatty acids, waxes, polyethylene-polyoxypropylene-block polymers, starches such as corn starch or potato starch, or combinations thereof

In some embodiments, the at least one additional compound is a diluent. As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

In some embodiments, the at least one additional compound is an excipient. As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient. Excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, dextran, fructose, mannose, lactose, galactose, sucrose, sorbitol, cellulose, methyl cellulose, hydroxypropyl methyl cellulose (hypromellose), glycerin, polyvinyl alcohol, povidone, propylene glycol, serum, amino acids, polyethylene glycol, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

In some embodiments, the at least one additional compound is any additional therapy that may be used for treating or inhibiting a viral, fungal, or bacterial infection, such as a SARS-CoV-2 infection, or a sequela thereof. For example, the additional therapy may be an anti-inflammatory compound, an immunotherapy, an analgesic, or any other suitable therapy. As used herein, an anti-inflammatory compound refers to compounds for the treatment of inflammation. Anti-inflammatory compounds include, for example, non-steroidal anti-inflammatory drugs (NSAIDs; such as aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, or nimesulide), corticosteroids (prednisone, betamethasone, budesonide, cortisone, dexamethasone, hydrocortisone, methylprednisolone, prednisolone, tramcinolone, or fluticasone), rapamycin, high density lipoproteins (HDL) or HDL-cholesterol elevating compounds (rosiglitazone), rho-kinase inhibitors, anti-malarial agents (hydroxychloroquine or chloroquine), acetaminophen, glucocorticoids, steroids, beta-agonists, anticholinergic agents, methyl xanthines, gold injections (sodium aurothiomalate), sulphasalazine, penicillamine, anti-angiogenic agents, dapsone, psoralens, anti-viral agents, or statins.

The presence, amount, quantity, and proportion of the at least one additional compound may depend upon the formulation of the composition, whether for topical, oral, intravenous, or other formulation type. The presence, amount, quantity, and proportion of the at least one additional compound may depend on the type of wound, the severity of the wound, the severity of pain, infection, or inflammation, or the intended treatment. Thus, the amount of at least one additional compound may be, for example, an amount ranging from 0.001% to 90%, such as 0.001, 0.005, 0.01, 0.05, 0.1, 0.5, 1, 2, 3, 4, 5, 10, 15, 20, 25, 30, 35, 40, 50, 60, 70, 80, or 90 percent, or an amount within a range defined by any two of the aforementioned values.

Where a range of values is provided, it is understood that the upper and lower limit, and each intervening value between the upper and lower limit of the range is encompassed within the embodiments.

The term “% w/w” or “% wt/wt” as used herein has its ordinary meaning as understood in light of the specification and refers to a percentage expressed in terms of the weight of the ingredient or agent over the total weight of the composition multiplied by 100. The term “% v/v” or “% vol/vol” as used herein has its ordinary meaning as understood in the light of the specification and refers to a percentage expressed in terms of the liquid volume of the compound, substance, ingredient, or agent over the total liquid volume of the composition multiplied by 100.

The invention is generally disclosed herein using affirmative language to describe the numerous embodiments. The invention also includes embodiments in which subject matter is excluded, in full or in part, such as substances or materials, method steps and conditions, protocols, or procedures.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1

### Isolation of SMS Cells for Formulating a Composition

The following example demonstrates the methods of isolating SMS cells for use in the compositions provided herein.

Small mobile stem (SMS) cells are grown in T25 flask using growth medium (37° C. and 5% CO2). The SMS cell population may contain a heterogeneous cell population of undifferentiated SMS cells and SMS derived differentiated cells.

SMS undifferentiated cells are present as a floating and adherent fraction. The floating fraction is predominantly undifferentiated SMS cells.

Floating undifferentiated SMS cells, distinguishable through their unique characteristic morphology, as explained previously, are obtained from the medium of the SMS cells grown in T25 flask.

Undifferentiated SMS cells can be isolated by differential centrifugation, removing clumps of cells or differentiated cells at low centrifugation speed followed by centrifuging undifferentiated SMS cells at high speed. Alternatively, the undifferentiated cells may be isolated by filtration, including differential filtration using filters having progressively smaller pore sizes to a pore size of 3-5 μm. The isolated undifferentiated SMS cells are examined under the microscope for homogeneity.

Undifferentiated SMS cells are grown in polypropylene tube (such as the bioreactor tubes: 15 ml provided by the manufacturer Techno Plastic Products AG, TPP).

The following example of growth medium is used: high sugar basal medium (Dulbecco's Modified Eagle Medium (DMEM), [+] 6g/L D-glucose, [−] sodium pyruvate, [−] L-glutamine, [−] Phenol red), to which 1% GlutaMAX™-I (100×), 10% calf serum, and 5 μg/mL human insulin was added. Alternatively a medium not containing any calf serum can be used. The cells are suspended occasionally by swirling.

Complete medium is replaced every week by centrifuging the SMS cells at 4200g for 15 min. The centrifugation may be varied at 3000g, 3500g, 4000g, 4100g, 4200g, 4300g, 4500g, or 5000g or by centrifugation at a speed that is within a range defined by any two of the aforementioned speeds, and the time adjusted accordingly.

Under these conditions, the volume size of the medium (cell crowdedness) is growth limiting to SMS cells. The SMS cell homogeneity is assessed microscopically and the SMS cell count is estimated by assessing spectroscopically turbidity of the suspension and/or by measuring the size of the pellet after centrifugation at high speed.

The SMS cell growth potential is assessed by inoculating cells into a new tube with growth medium. SMS cells grow mainly as individual cells not as clumps and remain under this condition mainly undifferentiated. The suspension culture is scalable such that increasing the volume of the medium increases the number of cells obtained.

### Example 2

### Treatment of Viral Infection using Compositions

The following example demonstrates the use of the compositions provided herein for treating a coronavirus infection.

The SMS cells isolated in Example 1 are aerosolized to generate an aerosol composition, formulated as an inhalant, intranasal formulation, or prepared as an intravenous formulation. Critically ill patients with SARS-CoV-2 are selected, as those who test positive for COVID-19 infection (as verified by RT-PCR testing or antibody testing). Subjects are randomized into control groups, test groups, and groups of different concentrations of SMS.

Treatment period is for up to one week, with subjects treating by seven daily administrations of aerosolized SMS or by intravenous therapy. Periodic evaluation for over 60 days after treatment termination is performed, including daily on days 1 through 7 post-treatment; twice weekly on days 8 through 14 post treatment; weekly on weeks 3 through 4 post treatment; and every two weeks for the remainder of the evaluation period.

The subjects are evaluated based on several outcomes, including: time to clinical improvement measured by time to weaning from mechanical ventilation, time to cessation of ICU monitoring, and time to cessation of vasopressor administration; mortality rate; frequency and characterization of adverse events and severe adverse events; tolerability; pulmonary function testing; lymphocyte count; complete blood count; routine chemistries; and/or radiologist chest imaging readings.

Subjects treated with the compositions described herein exhibit improved survival and improved symptoms associated with the viral infection.

### Example 3

### SMS Cell Therapy Animal Safety Testing

The acute toxicity of a formulation of living SMS cells suspended in Ringer's lactate solution with 5% rat serum after a repeat dose via intravenous administration was assessed. This study was performed according to the below Study Protocol and any applicable standard operating procedures.

A total of two (n=2) Sprague Dawley male rates were used. Animals were arbitrarily assigned to test and control group; one animal each. Test and control articles were prepared and administered intravenously slowly at a rate approximately 0.1 mL/sec via tail vein using a 25G needle. Test article: living SMS cells suspended in Ringer's lactate solution (B. Braun #L7500) with 5% rat serum (Sigma #R9759). Control article: Ringer's lactate solution (B. Braun #L7500) with 5% rat serum (Sigma #R9759). The test and control articles were administered on Day 1, Day 3, and Da 5, and the initial body weight was used for calculation of each dose volume.

The animals were observed individually at least three times on the day of dosing and at least once daily after the last dose. Animals were weighed on the day of dosing (Day 1), Day 8, and Day 14 and body weights were monitored. At the end of the study (Day 15), animals were euthanized, weighed, and gross necropsies were performed. At termination (Day 15), blood samples (1 mL) via cardiac puncture was collected and the sample was processed to plasma. Plasma was stored frozen until used.

Results: All animals appeared healthy during the course of the study and no biologically significant abnormalities were observed in any of the tested animals. Both test and control animals exhibited slight body weight loss after dosing but overall body weight gain on Day 8 and Day 14. No abnormalities were noted during gross necropsies.

Conclusion: The test and control substance were well tolerated when administered intravenously at a volume approximately 0.2 mL per animal on Day 1, Day 3, and Day 5. No toxic signs were observed in any of the tested animals and all animals gained weight at the end of the in-life portion of the study. All major organs appeared healthy at necropsy. A summary of the results are provided in Tables 1-3.

TABLE 1
Clinical Observations

| Day | Observations |
|---|---|
| 1 | Both animals appeared healthy |
| 2 | Both animals appeared healthy. Slight body weight loss in both |
| | animals. |
| 3 | Both animals appeared healthy. Slight body weight loss in both |
| | animals. |
| 4 | Both animals appeared healthy. Slight body weight loss in both |
| | animals. |
| 5 | Both animals appeared healthy. Slight body weight loss in test |
| | group animal. |
| 6 | Both animals appeared healthy. Slight body weight loss in test |
| | group animal. |
| 7 | Both animals appeared healthy. Slight body weight loss in test |
| | group animal. |
| 8 | Both animals appeared healthy. |
| 9 | Both animals appeared healthy. |
| 10 | Both animals appeared healthy. |
| 11 | Both animals appeared healthy. |
| 12 | Both animals appeared healthy. |
| 13 | Both animals appeared healthy. |
| 14 | Both animals appeared healthy. |
| 15 | Both animals appeared healthy. |

TABLE 2
Individual Animal Weights

| | Body weight | Body Weight |
|---|---|---|
| Day | (g) Test | (g) Control |
| Initial (pre-dose) | 454.2 | 415.3 |
| Day 2 | 451.2 | 411.2 |
| Day 3 (pre-dose) | 453.3 | 411.7 |
| Day 4 | 444.3 | 410.1 |
| Day 5 (pre-dose) | 450.3 | 419.2 |
| Day 6 | 446.1 | 418.2 |
| Day 7 | 446.6 | 419.6 |
| Day 8 | 457.0 | 426.9 |
| Day 9 | 456.9 | 427.6 |
| Day 10 | 458.6 | 432.1 |
| Day 11 | 463.4 | 436.5 |
| Day 12 | 466.7 | 435.4 |
| Day 13 | 456.6 | 446.8 |
| Day 14 | 462.6 | 448.9 |
| Day 15 | 469.5 | 448.2 |
| Body weight change (g) | +15.3 | +32.9 |
| Day 15 − Day 1 | | |

TABLE 3
Individual Observations at Necropsy

| Animal Number | Sex | Findings |
|---|---|---|
| 1 | M | No abnormalities observed |
| 2 | M | No abnormalities observed |

### Example 4

### Interaction of SMS Cells with Mesenchymal Stem Cells

### SMS Cell Binding to Human Adipose Derived Mesenchymal Stem Cells

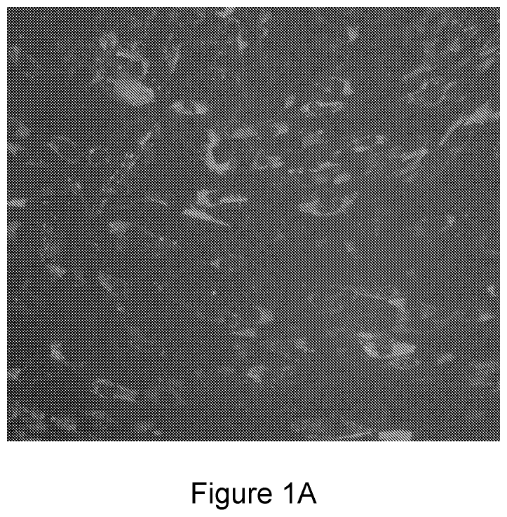

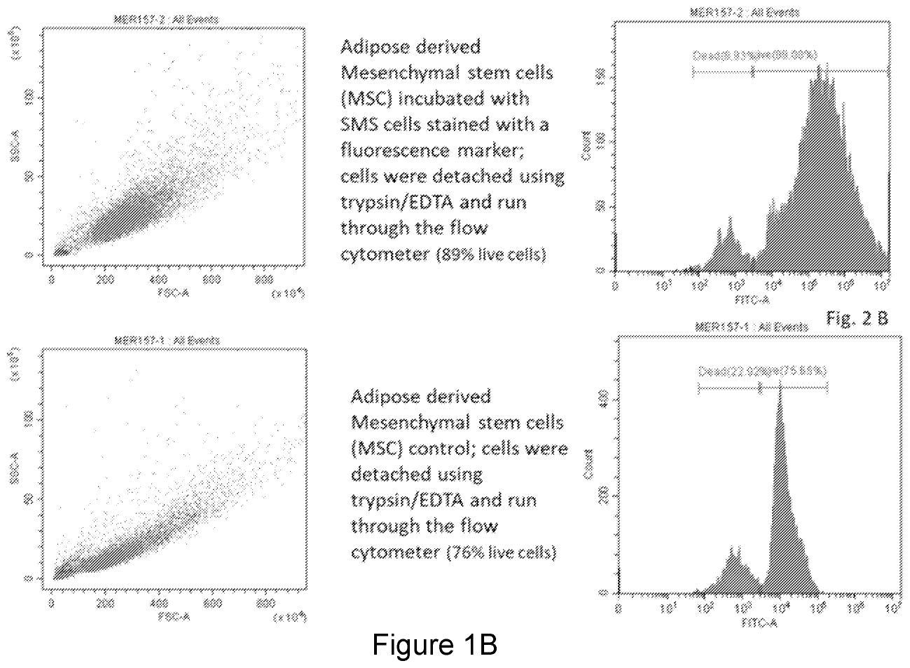

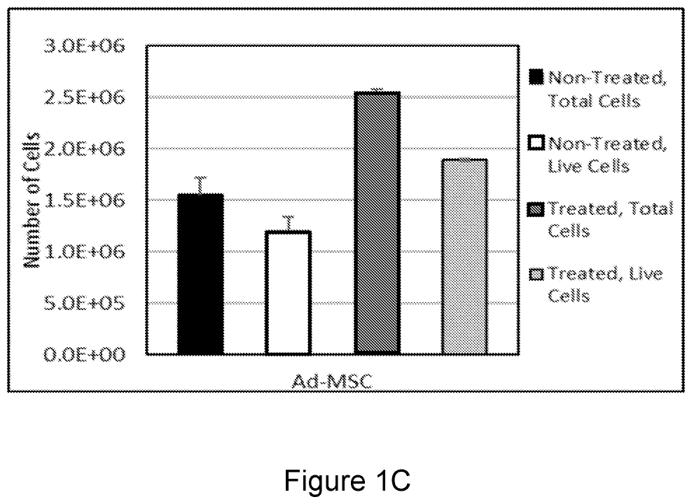

Commercially available human adipose derived mesenchymal stem primary cells were purchased from ThermoFisher Scientific (StemPro™ Human Adipose-Derived Stem Cells, Gibco, #R7788115). Human adipose derived mesenchymal stem cells frozen in liquid nitrogen were thawed and cultured at density of 2100 cells/cm² in 24 well culture plates using DMEM supplemented with 10% human serum and 0.2% antibiotics (gentamicin/amphotericin) for 5 days. Fluorescent stained SMS cells were added to the cultured adherent human adipose derived mesenchymal stem cells. The co-culture was incubated overnight, and the wells were washed with growth medium. Images were taken using an inverted fluorescence microscope. The results indicate strong adherence of SMS cells to human adipose derived mesenchymal stem cells based on fluorescence (FIG. 1A).

Adherent human adipose derived mesenchymal stem cells with attached SMS cells were detached from the well surface using a standard trypsinization protocol, and run through a flow cytometer detecting FITC fluorescence (CytoFLEX). The results indicate strong and continuous attachment of SMS cells to human adipose derived mesenchymal stem cells, based on positive fluorescence of the human adipose derived mesenchymal stem cells (FIG. 1B). The attachment of the two cells persist despite detachment of human adipose derived mesenchymal stem cells using trypsinization.

### SMS Cell Stimulation of Human Ddipose Derived Mesenchymal Stem Cells

Commercially available human adipose derived mesenchymal stem primary cells were purchased from ThermoFisher Scientific (StemPro™ Human Adipose-Derived Stem Cells, Gibco, #R7788115). Human adipose derived mesenchymal stem cells frozen in liquid nitrogen were thawed and cultured at density of 2100 cells/cm² in 24 well culture plates using DMEM supplemented with 10% human serum and 0.2% antibiotics (gentamicin/amphotericin) for 5 days. To flasks designated as “SMS treated”, SMS cells were added to the human adipose derived mesenchymal stem cells after 2 days (54.5 hours). Human adipose derived mesenchymal stem cells, with (“treated”) SMS cells or without (control), were simultaneously harvested after 5 days prior to cell confluence through detachment using a standard trypsinization protocol. Cells harvested were counted using the Countess Automated Cell Counter (Invitrogen) with the proper gating parameters (8-48 μm). The results demonstrate that human adipose derived mesenchymal stem cells cultured under in vitro optimal conditions are further stimulated for growth and proliferation in the presence of SMS cells (FIG. 1C).

### Example 5

### Interaction of SMS Cells with Fibroblasts

### SMS Cell Binding to Human Fibroblast Cells

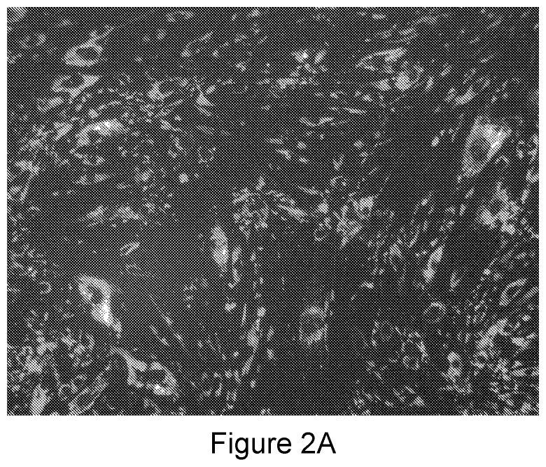

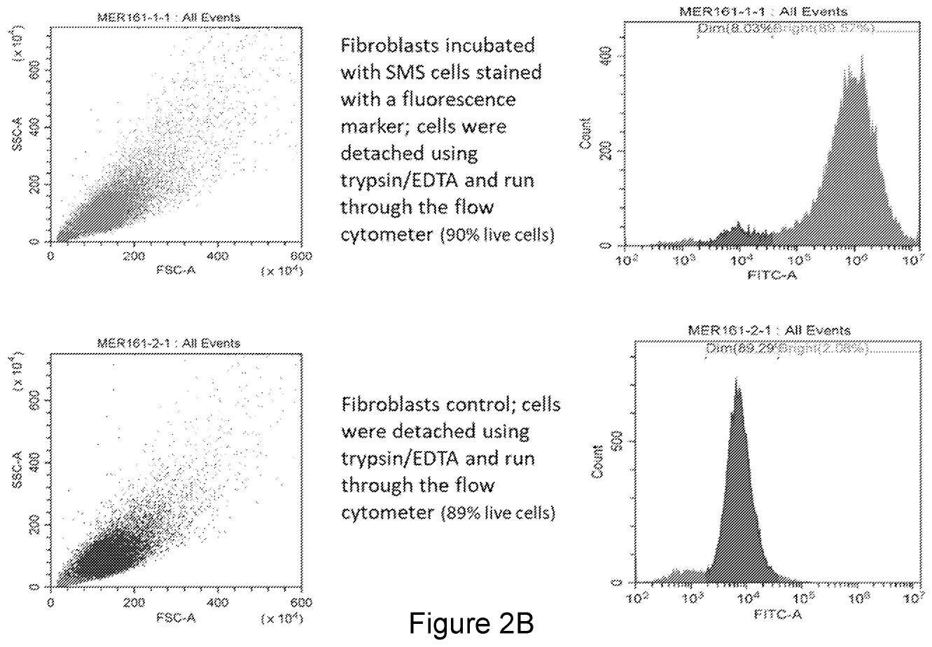

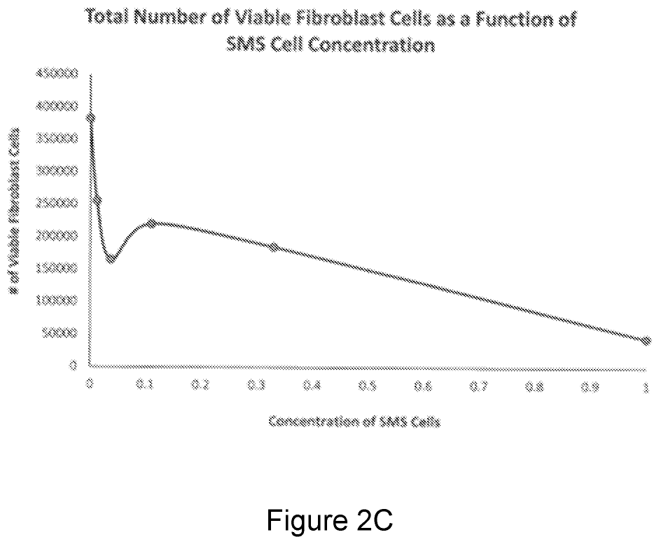

Commercially available human dermal fibroblasts were purchased from Lonza Scientific (CC-2511). Human derived fibroblast cells frozen in liquid nitrogen were thawed and cultured at a density of 2100 cells/cm² in 24 well culture plates using DMEM supplemented with 10% human serum and 0.2% antibiotics (gentamicin/amphotericin). Fluorescent stained SMS cells were added to the cultured fibroblasts. The co-culture was incubated overnight, and the wells were washed with growth medium. Images were taken with an inverted fluorescence microscope. The results indicate strong adherence of SMS cells to human dermal fibroblasts (FIG. 2A).

Adherent human fibroblasts with attached SMS cells were detached from the well surface using a standard trypsinization detachment protocol and run through a flow cytometer detecting FITC fluorescence (CytoFLEX). The results indicate strong and continuous attachment of SMS cells to human fibroblasts, based on the positive fluorescence of the human fibroblasts as indicated by flow cytometry (FIG. 2B). The attachment of the two cells persist despite detachment of human fibroblasts using trypsinization.

### SMS Cell Inhibition of Fibroblast Proliferation

Fibroblasts (Fb) were incubated under standard culturing conditions (37° C., 10% CO₂) in DMEM supplemented with 10% fetal bovine serum in a 48 well plate at a density of 1500 cells/cm². The medium was changed after 24 hours. SMS cells were added to the wells at various dilutions (1×,⅓, 1/9, 1/27, 1/81, and no cell control) performed in triplicate as depicted in Table 4. The co-culture was incubated for 4 days at standard culturing conditions (37° C., 10% CO₂) in DMEM supplemented with 10% fetal bovine serum. After incubation, cells were washed and detached using trypsinization and counted using the Countess Automated Cell Counter (Invitrogen). The results indicate significant inhibition of fibroblast proliferation that is proportional to the amount of SMS cell exposure (FIG. 2C).

TABLE 4
SMS cell and fibroblast concentrations tested

| SMS cell | | | | | | Control |
|---|---|---|---|---|---|---|
| concentration | 1x | 1/3 | 1/9 | 1/27 | 1/81 | (0x) |
| Fibroblast cell | Fb | Fb | Fb | Fb | Fb | Fb |
| density | 1500/cm² | 1500/cm² | 1500/cm² | 1500/cm² | 1500/cm² | 1500/cm² |
| Fibroblast cell | Fb | Fb | Fb | Fb | Fb | Fb |
| density | 1500/cm² | 1500/cm² | 1500/cm² | 1500/cm² | 1500/cm² | 1500/cm² |
| Fibroblast cell | Fb | Fb | Fb | Fb | Fb | Fb |
| density | 1500/cm² | 1500/cm² | 1500/cm² | 1500/cm² | 1500/cm² | 1500/cm² |

### Example 6

### Interaction of SMS Cells with Pulmonary Alveolar Epithelial Cells

### Isolation and Culture of Pulmonary Alveolar Type 2 Epithelial Primary Cells

Commercially available human pulmonary alveolar type 1 and 2 epithelial primary cells were purchased from ScienCell Research Laboratories (HPAEpiC, #3200). Human pulmonary alveolar type 1 and 2 epithelial primary cells frozen in liquid nitrogen were thawed. The contents of the vial was poured onto an equilibrated poly-L-lysine T75 culture flask using Alveolar Epithelial Cell Medium (ScienCell, AEpiCM, #3201) supplemented with 2% fetal bovine serum, 1% epithelial cell growth supplement (ScienCell, EpiCGS, #4152), 1% penicillin/streptomycin, in a 37° C., 5% CO₂ incubator. A seeding density of 10,000 cells/cm² was used. The medium was changed three time a week. Continuous culture for two weeks selected for proliferating alveolar type 2 cells and eliminated non-proliferating alveolar type 1 cells. Human pulmonary alveolar type 2 epithelial primary cells were detached from the culture vessel using trypsinization and frozen in cryopreservation medium.

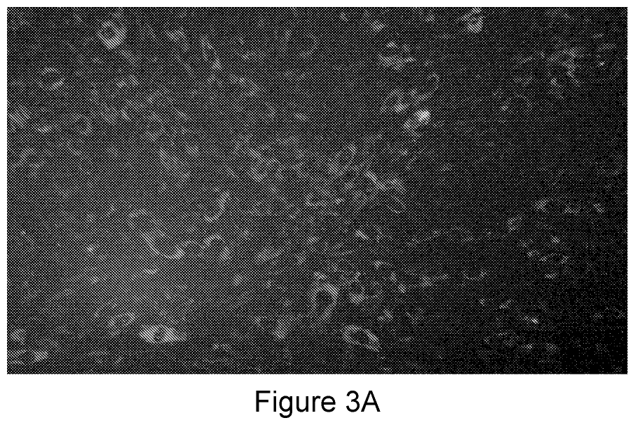

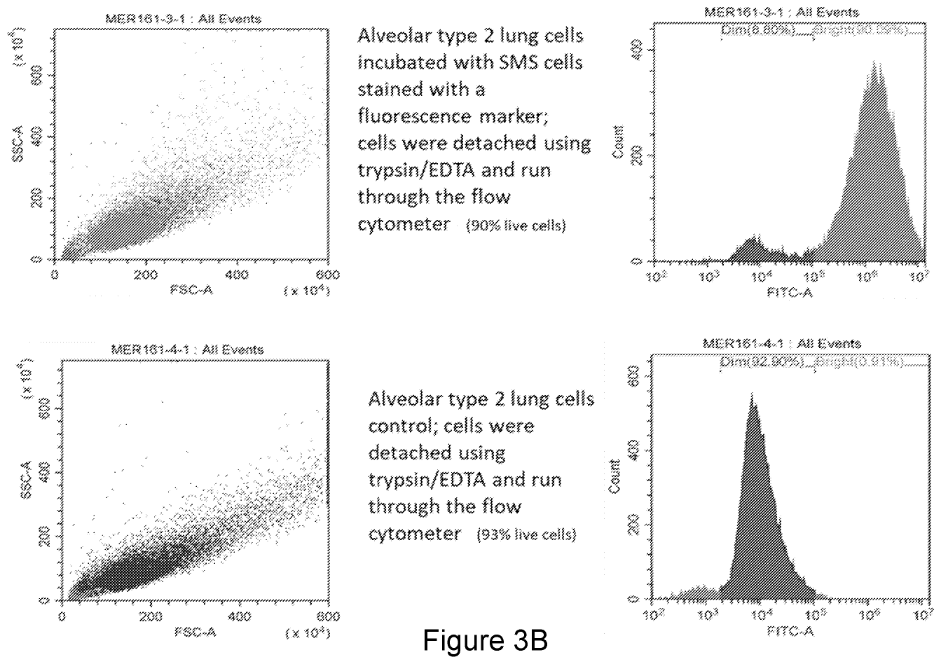

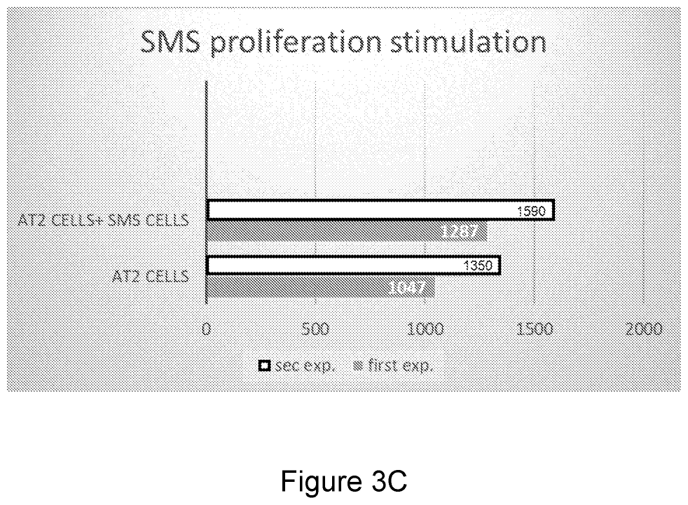

Fluorescent stained SMS cells were added to the isolated and cultured adherent human pulmonary alveolar type 2 epithelial primary cells. The co-culture was incubated overnight, and the wells were washed with growth medium. Images were taken using an inverted fluorescence microscope. The results indicate strong adherence of SMS cells to human pulmonary alveolar type 2 epithelial primary cells (FIG. 3A).

Adherent human pulmonary alveolar type 2 epithelial primary cells with attached SMS cells were detached from the well surface using a standard trypsinization detachment protocol and run through a flow cytometer detecting FITC fluorescence (CytoFLEX). The results indicate strong and continuous attachment of SMS cells to human pulmonary alveolar type 2 epithelial primary cells, based on the positive fluorescence of the human pulmonary alveolar type 2 epithelial primary cells as indicated by flow cytometry (FIG. 3B). The attachment of the two cells persist despite detachment of the pulmonary alveolar epithelial cells using trypsinization.

SMS Cell Stimulation of Human Pulmonary Alveolar Type 2 Epithelial primary cells in the absence of fetal bovine serum

Cryopreserved isolated human pulmonary alveolar type 2 epithelial primary cells were thawed in Alveolar Epithelial Cell Medium with 1% epithelial cell growth supplement in the absence of 2% fetal bovine serum.

To flasks of pulmonary alveolar type 2 epithelial cells designated as “SMS treated”, SMS cells were added after 3 days (74.5 hours). The cells, cultured with (“treated”) SMS cells or without SMS cells (control) were simultaneously harvested after 8 days, prior to cell confluence, through detachment using a standard trypsinization protocol. Cells harvested were counted using the Countess Automated Cell Counter (Invitrogen) with the proper gating parameters (8-60 μm). The results demonstrate that human pulmonary alveolar type 2 epithelial primary cells cultured under in vitro serum deprived conditions are stimulated for growth and proliferation in the presence of SMS cells (FIG. 3C).

Differential gene expression of human pulmonary alveolar type 2 epithelial cells in the presence of SMS cell stimulation in the absence of fetal bovine serum

Human pulmonary alveolar type 2 epithelial cells cultured with and without SMS cells (as described above) were used to detect differential gene expression. Cells were centrifuged and resuspended in pre-chilled PBS so that each 100 μL has ≥300,000 cells. The supernatant was removed without disturbing the pellet and placed into a -80° C. freezer for flash freezing. The pellet was shipped on dry ice for mRNA analysis to Active Motif. Total RNA of cells was isolated and individual RNAs were identified through sequencing and the relative quantities were assessed. Fold changes and shrunken loge fold changes (logFC) were computed using DESeq2 (available on the world wide web at bioconductor.org/packages/release/bioc/html/DESeq2.html). The results from RNA analysis of the human pulmonary alveolar type 2 epithelial cells demonstrate 764 genes that are upregulated and 546 genes that are downregulated by in vitro interaction with SMS cells (Table 5). From the top 40 genes that experienced the greatest magnitude in change between SMS cell treatment and control, 13 exemplary genes and their quantitative expression values are shown in Table 6. A negative value for the shrunken logFC of the ratio of non-treated to treated conditions indicates an increase in expression of the gene after SMS cell treatment, while a positive value for the shrunken logFC of the ratio of non-treated to treated conditions indicates a decrease in expression of the gene after SMS cell treatment. Additional information on DESeq2 can be found in Love et al. “Moderated estimation of fold change and dispersion for RNA-seq data with DESeq2” Genome Biology (2014) 15(12):550, which is hereby incorporated by reference in its entirety. Shrinking the logFC generates more accurate loge fold change estimates to account for low counts or high dispersion values but does not affect the outcome of significantly different gene expression.

Examples of genes that were upregulated in the pulmonary alveolar type 2 epithelial cells treated with SMS cells include Dickkopf-related protein 1 (DKK1), N-acylglucosamine 2-epimerase (RENBP), growth/differentiation factor 15 (GDF15), dermokine (DMKN), ferritin, light (FTL), tryptophan 2,3-dioxygenase (TDO2), apolipoprotein E (APOE), chitinase-3-like protein 1 (CHI3L1), and nuclear receptor subfamily 4 group A member 2 (NR4A2).

Examples of genes that were downregulated in the pulmonary alveolar type 2 epithelial cells treated with SMS cells include interleukin-11 (IL-11), SPRY domain-containing SOCS box protein 1 (SPSB1), cytochrome P450 26B1 (CYP26B1), and frizzled-8 (FZD8).

The modulated genes that were identified indicate that treatment with SMS cells have an effect on reducing inflammation, improving blood vessel diameter, reducing cell differentiation, increasing cell proliferation, and enhancing protection against pathogens.

TABLE 5
Differential gene expression in pulmonary alveolar
epithelial cells cultured with SMS cells

| | |
|---|---|
| Total Genes | 28395 |
| Genes with zero counts | 9446 |
| Genes with low counts | 6439 |
| Total genes used for testing | 12510 |
| Up-regulated genes: logFC(Non-treated/Treated) > 0 | 764 |
| Down-regulated genes: logFC(Non-treated/Treated) < 0 | 546 |
| Total differential genes (adjP < 0.1) | 1310 |

TABLE 6
Differential gene expression of exemplary genes

| | | | | | | Shrunken | p-value |
|---|---|---|---|---|---|---|---|
| | | | | | | logFC | adjusted |
| | | | | | | Non-treated/ | Non-treated/ |
| Gene | Length | SMS treated 1 | SMS treated2 | Non-treated 1 | Non-treated 2 | treated | treated |
| APOE | 1460 | 942 | 902 | 413 | 341 | −1.05 | 1.50293E−24 |
| CHI3L1 | 2196 | 2128 | 2035 | 762 | 730 | −1.34 | 2.53095E−71 |
| FTL | 871 | 92900 | 86827 | 50571 | 51855 | −0.79 | 2.11687E−98 |
| RENBP | 1471 | 139 | 142 | 45 | 48 | −0.68 | 2.21973E−06 |
| NR4A2 | 5439 | 817 | 793 | 469 | 478 | −0.64 | 3.84756E−10 |
| IL11 | 2378 | 109 | 105 | 263 | 256 | 0.77 | 2.51826E−08 |
| TDO2 | 1703 | 334 | 378 | 123 | 152 | −0.89 | 1.90231E−11 |
| FZD8 | 3186 | 133 | 147 | 381 | 366 | 0.95 | 1.06367E−13 |
| GDF15 | 1200 | 4890 | 4702 | 2903 | 3057 | −0.66 | 2.99963E−33 |
| DKK1 | 1805 | 1141 | 1215 | 664 | 752 | −0.64 | 1.01344E−11 |
| CYP26B1 | 4769 | 1222 | 1273 | 2653 | 2653 | 1.01 | 2.74627E−53 |
| SPSB1 | 3118 | 729 | 754 | 1609 | 1635 | 1.02 | 1.75977E−37 |
| DMKN | 3065 | 1074 | 1005 | 390 | 383 | −1.2 | 3.92647E−36 |

### Example 7

### Treatment of an Inflammatory Disease with SMS cells

A subject presents with an inflammatory disease. Some non-limiting examples of inflammatory disease include fibrosis, such as pulmonary fibrosis, chronic obstructive pulmonary disease (COPD), or inflammation as a result of a pathogenic infection such as a viral infection (e.g. a SARS-CoV-2 infection).

A pharmaceutical composition of SMS cells are administered to the subject enterally, orally, intranasally, parenterally, subcutaneously, intramuscularly, intradermally, or intravenously. The pharmaceutical composition may include 10³, 10⁴, 10⁵, 10⁶, 10⁷, 10⁸, 10⁹, 10¹⁰, or 10¹¹ SMS cells, or any number of SMS cells within a range defined by any two of the aforementioned numbers. The pharmaceutical composition may be administered in 1, 2, 3, 4, 5, 6, 7, 8, 9, 10 doses, or at least 1, 2, 3 ,4, 5, 6, 7, 8, 9, or 10 doses. If there are multiple doses, these can be administered every 1, 2, 3, 4, 5, 6, 7, 8, 9, 10, 11, 12, 24, 36, or 48 days or weeks, or any time within a range defined by any two of the aforementioned times, in between doses.

The subject is monitored for an improvement in the inflammatory conditions or symptoms thereof following administration of the pharmaceutical composition of SMS cells.

In some embodiments, the pharmaceutical composition of SMS cells may be administered along with another standard of care treatment. For example, if the inflammatory disease is related to a viral infection, the subject may be administered an anti-viral therapy, such as dexamethasone, convalescent serum, tocilizumab, sarilumab, ribavirin, favipiravir, darunavir, galidesivir, interferon alpha, interferon beta, lopinavir, ritonavir, remdesivir, triazavirin, umifenovir, or other anti-viral standard of care.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of promoting or improving repair or regeneration of damaged tissue or for improving recovery from a viral, bacterial, or fungal infection or a sequela thereof, comprising:
selecting a subject that has or has had a viral, bacterial, or fungal infection or the sequela thereof; and
administering to the selected subject a composition comprising a therapeutically effective amount of small mobile stem (SMS) cells.

2. The method of claim 1, wherein the SMS cells are obtained from peripheral blood.

3. The method of claim 1, wherein the SMS cells are allogeneic to the subject.

4. The method of claim 1, wherein the SMS cells are autologous to the subject.

5. The method of claim 1, wherein the damaged tissue is lung tissue.

6. The method of claim 5, wherein administering the composition reduces or inhibits alveolar cell injury, reduces or inhibits respiratory endothelial cell injury, increases or improves repair of damaged lung tissue, increases or enhances regeneration of damaged lung tissue, or reduces development of pulmonary fibrosis, or any combination thereof.

7. The method of claim 1, wherein the damaged tissue is a result of the viral, bacterial, or fungal infection.

8. The method of claim 1, wherein the viral infection is caused by a single-stranded RNA virus, a double-stranded RNA virus, a positive-sense ssRNA virus, a negative-sense ssRNA virus, a double-stranded DNA virus, or a single-stranded DNA virus.

9. The method of claim 1, wherein the viral, bacterial, or fungal infection or the sequela thereof results from infection by a coronavirus, pox virus, smallpox virus, marburg virus, flaviviruses, influenza virus, parainfluenza virus, respiratory syncytial virus, rubeola virus, human immunodeficiency virus, human papillomavirus, varicella-zoster virus, herpes simplex virus, cytomegalovirus, an Epstein-Barr virus, JC virus, rhabdovirus, rotavirus, rhinovirus, adenovirus, papillomavirus, parvovirus, picornavirus, poliovirus, hantavirus, Filovirus, coxsackievirus, equine encephalitis virus, a Rift Valley fever virus, an alphavirus, hepatitis A virus, hepatitis B virus, hepatitis C virus, hepatitis D virus, or a hepatitis E virus, a pneumonia, Streptococcus pneumoniae, Staphylococcus aureus, Haemophilus influenazae, Moraxella catarrhalis, anaerobes, Gram-negative organisms, Mycoplasma pneumoniae, Chlamydia pneumoniae, Group A Streptococcus, Klebsiella pneumoniae, Legionella pneumophila, Streptococcus pyogenes, Streptococcus agalactiae, Peptostrepotoccus, Corynebacterium diphtheriae, Bordetella pertussis, Mycobacterium tuberculosis, Pseudomonas aeruginosa, Escherichia coli, Curvularia, Pneumocystis, Cryptococcus species, Histoplasma capsulatum, Aspergilli, Mucorales, Fusarium, Scedosporium, Penicillium, or any combination thereof.

10. The method of claim 9, wherein the coronavirus is a MERS-CoV, SARS-CoV, or SARS-CoV-2.

11. The method of claim 9, wherein the coronavirus is SARS-CoV-2.

12. The method of claim 1, wherein the composition comprises aerosolized SMS cells formulated for administration via inhalation.

13. The method of claim 1, wherein the composition is administered intravenously.

14. The method of claim 1, wherein the SMS cells are administered in combination with an antibiotic, an antifungal, a vaccine or an anti-viral medication.

15. The method of claim 14, wherein the vaccine is a vaccine against one or more of coronavirus, cholera, dengue, diphtheria, Haemophilus influenza type b infection, hepatitis A, hepatitis B, influenza, Japanese encephalitis, meningococcal meningitis, pertussis, polio, rabies, tetanus, tuberculosis, typhoid, or yellow fever.

16. The method of claim 14, wherein the vaccine is a coronavirus COVID-19 vaccine.

17. The method of claim 1, wherein administration of the composition to the selected subject upregulates Dickkopf-related protein 1 (DKK1), N-acylglucosamine 2-epimerase (RENBP), growth/differentiation factor 15 (GDF15), dermokine (DMKN), ferritin, light (FTL), tryptophan 2,3-dioxygenase (TDO2), apolipoprotein E (APOE), chitinase-3-like protein 1 (CHI3L1), or nuclear receptor subfamily 4 group A member 2 (NR4A2), or any combination thereof, in the selected subject.

18. The method of claim 1, wherein administration of the composition to the selected subject downregulates interleukin-11 (IL-11), SPRY domain-containing SOCS box protein 1 (SPSB1), cytochrome P450 26B1 (CYP26B1), or frizzled-8 (FZD8), or any combination thereof, in the selected subject.

19-41. (canceled)

42. A method of treating or inhibiting an inflammatory disease, comprising:
selecting a subject having the inflammatory disease; and
administering to the subject a composition comprising a therapeutically effective amount of small mobile stem (SMS) cells.

43. The method of claim 42, wherein the inflammatory disease is a pulmonary inflammatory disease.

44. The method of claim 42, wherein the inflammatory disease comprises fibrosis, such as pulmonary fibrosis, liver fibrosis, kidney fibrosis, or cardiac fibrosis.

45. The method of claim 42, wherein the inflammatory disease comprises chronic pulmonary obstructive disease (COPD).

46-54. (canceled)

55. The method of claim 42, wherein the composition comprises aerosolized SMS cells.

56. The method of claim 42, wherein the composition is formulated for administration by inhalation.

57. The method of claim 42, wherein the composition is formulated for intravenous administration.

58. The method of claim 42, wherein the composition is administered with a standard of care treatment for the inflammatory disease.

59-63. (cancelled)
